SEQUENCE LISTING (28 sequences)

SEQ ID NO: 1 - DNA, 2253 bp, Streptococcus agalactiae
gtgattatcgatcgactgttacaaagaagccactctcatctaccgattctacaagctacatttggcttagagagagaaagccttcgtattcaccaacccactcaaagggttgctcaaacaccccatcccaaaacattagggagtcgtaactatcatccttatatccaaacggattatagtgaacctcaattagaactcattacacctattgcaaaggatagccaagaagccattcgatttttgaaagctataagtgatgttgctggacgctctatcaaccacgatgaatacttatggccgctttctatgccacccaaagtgagggaagaagacatacagattgcccaattagaggatgcttttgaatatgattatcgcaaatatttggagaagacttatggaaaactaatacaatccatctctggaattcactataatctcggtttaggtcaagagttattaacttctctctttgaattgagtcaagcagataatgctattgattttcagaaccagctttacatgaaattgtctcaaaattttttacgttaccgttggttactaacttatctatacggagcgagcccagtagctgaagaagactttttagatcagaaactgaataaccctgtccggtcacttagaaatagtcacttaggttatgtgaatcataaagatattcgtatttcttatactagcttgaaggattatgtcaacgatctagaaaatgctgtaaaaagtgggcaattgattgctgagaaagaattttattcacctgttcgtctccgtggtagtaaggcctgtcgtaattatttagaaaaaggaattacatatttagagtttcgcacttttgatctcaacccatttagcccaatcggtataacgcaggaaactgttgatactgttcaccttttcttattggcgcttttgtggatagattctagtagtcatattgaccaagatatcaaggaagccaatagattaaatgaccttatagcacttagtcatccattagaaaaattacctaatcaagcaccagtttctgacttagtggatgctatgcaatctgttatccaacattttaacttatcaccttactatcaagacctattggaatctgtaaaaaggcaaattcagtcacctgaattaacggtagctggtcaacttttagaaatgattgaaggactttccttagaaacatttggacaaagacaaggacaaatttatcacgattatgcttgggaagctccctacgccttaaagggatatgaaacgatggaactttctacccaattgctactatttgacgttatccaaaaaggagtcaacttcgaagtgctggatgaacaagatcaattcctaaaattatggcacaatagtcatattgagtacgttaaaaatggtaatatgacctcaaaagataactatattgtaccgctcgctatggctaacaaagttgttacaaaaaaaatcctagatgaaaagcatttcccaactccttttggagatgaatttactgaccgtaaagaggcacttaactatttttcacaaatccaagataaaccaatcgtcgttaagccaaaatctacaaactttggtttaggcatttctatttttaaaacatcagctaacttagcttcttatgaaaaagcaattgacatcgcttttaccgaagatagtgctattcttgtggaagaatatattgagggtaccgaataccgtttcttcgtccttgagggggactgtattgctgtattgcttagggtagcagctaatgtcgttggagatggtattcatactattagtcagctagttaaacttaaaaaccaaaaccctctcagaggctatgatcatcgctctcctctagaggttattgagctcggagaagtagaacagttaatgttggaacaacaaggatacactgttaacagtatccctccagaaggaacaaaaatagaacttcgccgtaattctaatatttccactggcggtgactctatagatgtaacaaatacaatggatcctacatataaacaactagcagctgagatggcagaggctatgggggcttgggtttgtggagttgatttaattattcctaatgccactcaagcatactcaaaagataagaaaaatgctacttgcattgaactaaactttaacccattgatgtatatgcacacctactgtcaagaggggcctggtcaatctattacacctcgaattttggctaaactgttcccagaattataa

SEQ ID NO: 2 - protein, 750 aa, Streptococcus agalactiae
MIIDRLLQRSHSHLPILQATFGLERESLRIHQPTQRVAQTPHPKTLGSRNYHPYIQTDYSEPQLELITPIAKDSQEAIRFLKAISDVAGRSINHDEYLWPLSMPPKVREEDIQIAQLEDAFEYDYRKYLEKTYGKLIQSISGIHYNLGLGQELLTSLFELSQADNAIDFQNQLYMKLSQNFLRYRWLLTYLYGASPVAEEDFLDQKLNNPVRSLRNSHLGYVNHKDIRISYTSLKDYVNDLENAVKSGQLIAEKEFYSPVRLRGSKACRNYLEKGITYLEFRTFDLNPFSPIGITQETVDTVHLFLLALLWIDSSSHIDQDIKEANRLNDLIALSHPLEKLPNQAPVSDLVDAMQSVIQHFNLSPYYQDLLESVKRQIQSPELTVAGQLLEMIEGLSLETFGQRQGQIYHDYAWEAPYALKGYETMELSTQLLLFDVIQKGVNFEVLDEQDQFLKLWHNSHIEYVKNGNMTSKDNYIVPLAMANKVVTKKILDEKHFPTPFGDEFTDRKEALNYFSQIQDKPIVVKPKSTNFGLGISIFKTSANLASYEKAIDIAFTEDSAILVEEYIEGTEYRFFVLEGDCIAVLLRVAANVVGDGIHTISQLVKLKNQNPLRGYDHRSPLEVIELGEVEQLMLEQQGYTVNSIPPEGTKIELRRNSNISTGGDSIDVTNTMDPTYKQLAAEMAEAMGAWVCGVDLIIPNATQAYSKDKKNATCIELNFNPLMYMHTYCQEGPGQSITPRILAKLFPEL

SEQ ID NO: 3 - DNA, 2265 bp, Streptoccus mutans
atgcacatcaatcaattattacagcatgcaaattctgacttaccccttcttcaagctaattttggtttagaaagagaaagtctccgtatcaataaaacaaatcaccggctggctcagacacctcatccaacagcactgggttctcgccagtttcatccttatattcaaacagattacagtgagtctcagatggaactaatcacgcctgtcgctcattccagcaaggaagtccttcgttttttaggggctattactgatgttgcagagcgcagtattgaccaaaaccaatacctttggcctttgtctatgccgcctcaaattacagaagacgaaattgaaattgcccagttagaagatgactttgaattttcctatcgtcagtatttagataaaaaatatggaaaaatcctgcaatccatatctggcattcattataacatggagctaggtgctgatttaatgaatgaactttttgaacttagcggttatcagtctttcattgactttaaaaatgatctctacttaaaagtggctcagaatttcttaaactatcgttggttcctgacctacctttatggggctagtcccttggctgaaaaaggatttttaaatgaagagctcagccaaactgttcgctcaatccgaaacagtcatttaggctatgtcaatactgatgatattaaggttcctttcgacagtctcgaaaattatatctcaagtattgagcactacgttaaaagcggtgctctatcagctgaaaaagaattctattcagctgttcgtttgcgtggcagtaagcataatcgtgactatcttactaaaggaatcacttatttagaatttcgctgttttgatcttaatcccttcaataatcgcggcattacgcaagaaaccattgacagtgtccatctctttatcttggccatgctctggcttgacacaccaaaaaagctgaatcaagcacttgataaggctcaaaaacttaatgataaaattgcattaagccatcctctggaaaaattaccaaaggagaactctgcttcccttattatagaagcaatggaagccttaattaaacactttaaattaccaagttactatgatgatttactaattgctatcaaaaaacaagttgaaaatcctaagttaactctaagcggccgtctctttgagcatattaagcatgcctcattggagcattttggacagaaaaaagggcaagattatcataactacgcttggcaaaattattatgccctcaagggctatgaaaatatggaattgtcaacacaaatgctgctttttgataccatccaaaaaggaattcattttgaaattttagatgaaaatgatcaatttctcaaactgtggcataatgatcatattgaatatgtcaaaaatggcaacatgacttctaaagataactacgttatccccctagccatggccaataaagtggtgactaaaaaaatactaagagaaaacggctaccctgtcccagcaggagctgaatttgacaataaagacgaggccctccgctattattcccaaatcaaaaataaacctattgttgttaaacctaagacaaccaatttcggacttgggatttctatttttgaaacagcagccagtcacaatgattacgaaaaagcacttgacattgcctttattgaagactattcggtccttgtggaagaatttataccaggaacagaataccgtttcttcattctggacggtaaatgtgaggccgttctcttgcgggttgcggccaacgtcgttggagacggccatagtactgttcggcagctcgtggcacaaaaaaatagggatcctcttcgtggtcgggagcatcgctctccacttgaaatcattgatctcggtgatattgaattgctgatgctgcagcaagaaggttataccctagaagacatcttaccaaagggcaagaaggtgaatcttcgtggtaattccaatatctcaactggcggcgattcgattgatgtgacagaaaccatggatcctagctacaaacaactagcagctaatatggcgacagcaatgggagcttgggtttgtggggtggatttgatcattccagataccaacttaaaagccagcaagggaaagccaaactgtacctgcattgagctcaatttcaatccatccatgtatatgcatacctattgctaccaaggaccgggacaagttatcacaggcaaaattctagccaagctctttcctgaaataagcaccaagatataa

SEQ ID NO: 4 - protein, 754 aa, Streptococcus mutans
MHINQLLQHANSDLPLLQANFGLERESLRINKTNHRLAQTPHPTALGSRQFHPYIQTDYSESQMELITPVAHSSKEVLRFLGAITDVAERSIDQNQYLWPLSMPPQITEDEIEIAQLEDDFEFSYRQYLDKKYGKILQSISGIHYNMELGADLMNELFELSGYQSFIDFKNDLYLKVAQNFLNYRWFLTYLYGASPLAEKGFLNEELSQTVRSIRNSHLGYVNTDDIKVPFDSLENYISSIEHYVKSGALSAEKEFYSAVRLRGSKHNRDYLTKGITYLEFRCFDLNPFNNRGITQETIDSVHLFILAMLWLDTPKKLNQALDKAQKLNDKIALSHPLEKLPKENSASLIIEAMEALIKHFKLPSYYDDLLIAIKKQVENPKLTLSGRLFEHIKHASLEHFGQKKGQDYHNYAWQNYYALKGYENMELSTQMLLFDTIQKGIHFEILDENDQFLKLWHNDHIEYVKNGNMTSKDNYVIPLAMANKVVTKKILRENGYPVPAGAEFDNKDEALRYYSQIKNKPIVVKPKTTNFGLGISIFETAASHNDYEKALDIAFIEDYSVLVEEFIPGTEYRFFILDGKCEAVLLRVAANVVGDGHSTVRQLVAQKNRDPLRGREHRSPLEIIDLGDIELLMLQQEGYTLEDILPKGKKVNLRGNSNISTGGDSIDVTETMDPSYKQLAANMATAMGAWVCGVDLIIPDTNLKASKGKPNCTCIELNFNPSMYMHTYCYQGPGQVITGKILAKLFPEISTKI

SEQ ID NO: 5 - DNA, 2274 bp, Pasteurella multocida
atgaaaattcaacatatcattcatgaaaaccaattaggattattgtttcaacaaggctcattcgggttagaaaaagaaagtcagcgcgtgactgccgatggtgcaattgtaacgactcctcatcctgcggtgtttggtaatcgccgttatcatccttatattcaaacagattttgctgagagccaacttgagctgattaccccgccaactaaaaagttagaagatacgtttcgttggttgtctgttattcatgaagtggtacaacgttcgctacccgaagaagaatatattttcccattgagtatgcctgcgggattgccagcagaagagcaaattcgtgtggcacaattagataacccagaagacgtggcttatcgtgaatatttagtgaaaatatatggtaaaaacaagcagatggtcagtgggattcactacaactttcagctttcccccgacttgatcacacgtttattccgcttgcagaatgagtatcaaagtgcggtcgattttcagaatgatttatatctgaaaatggcaaaaaacttcttacgttatcaatggattttgttgtatctattagcggcaacaccaacggttgaatccgcttattttaaagatggatccccgttagcgaaagggcaattcgtgcgtagtttacgttcaagtcaatatggttatgtgaatgatcccgaaattaacgtttcttttgatagtgtcgaaaagtatgttgaaagcttagagcattgggtatcaacggggaaattgattgcagaaaaagaattttattctaatgtgcgtttgcgtggtgcaaagaaagcgcgcgaatttttgacaacgggtattcaatatctggagttccgtttatttgacttaaatccgtttgaaatttacgggattagcctaaaagatgcgaagtttatccacgtgttcgccttattcatgatttggatggatcacactgctgatcaagaagaagtggaattaggtaaagcgcgtttggcagaagtggcttttgagcatccattagaaaaaaccgcttatgccgtcgaaggcgaattggtgctcttagaattattgtcgatgctagagcaaattggcgcagagccggaattatttgagattgtgaaagagaaattaactcaattcacggatccaagcaaaacagtggcaggacgtttagtacgcgcgattgagcaagccgggagcgatcaacaattaggtgcccagctcgcacagcaatataaagcccaagcctttgaacgtttttatgccttatctgcattcgataacatggagctttctacccaagcgttgttatttgatgtgattcagaaagggattcatactgagattttggatgaaaatgatcaattcttgtgcttaaaatatggagatcatattgagtatgtgaaaaacggcaatatgacctcacacgacagttatatttcaccgctaattatggaaaataaagtggtgaccaaaaaagtattgcaaaaagcgggctttaatgtgccacaaagtgtggagtttacttctcttgagaaagcggtggcaagttatgcactttttgaaaaccgtgcggtagtgattaaacccaaatccacgaattatggcttgggtatcaccattttccaacaaggcgtgcaaaaccgtgaggattttgctaaggcgctagaaattgctttccgtgaagataaagaagtaatggtggaagattatttagtcggaacagaataccgcttctttgtattaggtgatgaaacattggcggtattattgcgtgtaccagccaatgtcgtaggagacagtgtgcattctgtggcagaacttgttgccatgaaaaatgatcatccgctacgaggtgatggtagccgtacaccattgaagaaaattgctttaggcgaaattgagcagttacagctcaaggagcaaggtttaaccattgatagcattcccgctaaagatcagcttgtacaactacgagccaattccaatattagtacaggtggtgattccattgatatgacggatgaaatgcacgagagttataaacaactggctgtcggtatcacgaaagcgatgggcgcggcggtgtgtggtgtggatttaattatccctgacttgaagcaaccggctacgccaaacttaacttcttggggtgtgattgaagcgaattttaatccgatgatgatgatgcatattttcccttatgcgggtaaatctcgtcgtttaactcagaatgtgattaagatgctctttcctgaactggagtaa

SEQ ID NO: 6 - protein, 757 aa, Pasteurella multocida
MKIQHIIHENQLGLLFQQGSFGLEKESQRVTADGAIVTTPHPAVFGNRRYHPYIQTDFAESQLELITPPTKKLEDTFRWLSVIHEVVQRSLPEEEYIFPLSMPAGLPAEEQIRVAQLDNPEDVAYREYLVKIYGKNKQMVSGIHYNFQLSPDLITRLFRLQNEYQSAVDFQNDLYLKMAKNFLRYQWILLYLLAATPTVESAYFKDGSPLAKGQFVRSLRSSQYGYVNDPEINVSFDSVEKYVESLEHWVSTGKLIAEKEFYSNVRLRGAKKAREFLTTGIQYLEFRLFDLNPFEIYGISLKDAKFIHVFALFMIWMDHTADQEEVELGKARLAEVAFEHPLEKTAYAVEGELVLLELLSMLEQIGAEPELFEIVKEKLTQFTDPSKTVAGRLVRAIEQAGSDQQLGAQLAQQYKAQAFERFYALSAFDNMELSTQALLFDVIQKGIHTEILDENDQFLCLKYGDHIEYVKNGNMTSHDSYISPLIMENKVVTKKVLQKAGFNVPQSVEFTSLEKAVASYALFENRAVVIKPKSTNYGLGITIFQQGVQNREDFAKALEIAFREDKEVMVEDYLVGTEYRFFVLGDETLAVLLRVPANVVGDSVHSVAELVAMKNDHPLRGDGSRTPLKKIALGEIEQLQLKEQGLTIDSIPAKDQLVQLRANSNISTGGDSIDMTDEMHESYKQLAVGITKAMGAAVCGVDLIIPDLKQPATPNLTSWGVIEANFNPMMMMHIFPYAGKSRRLTQNVIKMLFPELE

SEQ ID NO: 7 - DNA, 2331 bp, Listeria monocytogenes
atgataaaacttgatatgaccatgcttgattcttttaaagaagaccctaatctccggaagcttttattttctggacattttggtttagaaaaagaaaatattcgcgtaacttctgatggaaaattggcccttacgccacatccagctatttttggtccaaaagaggataacccatatattaaaaccgatttttcggaaagtcagattgaaatgattaccccggtgacggactcgattgactctgtatatgaatggcttgaaaatcttcataatatcgtttcgctgcgctccgaaaacgaactactttggccatctagcaatccgcccattttaccagcggaagaagatattccaattgctgaatataaaacacccgatagtccggacagaaaataccgcgaacatttagcaaaaggctacggtaaaaaaatccaattattatctggcattcattataatttctcattcccggaagctttaattgacggattatatgccaatattagcctcccagaagaatcgaagcaagattttaaaaatcgtctatatttaaaagttgcaaaatactttatgaaaaatcgctggttgcttatttatttaactggcgcaagtccggtttaccttgctgatttctcgaaaacgaagcacgaagaatcccttccagacggtagtagcgcgcttcgtgatggaatttctcttcgaaatagtaatgctgggtataaaaacaaagaagctcttttcgttgattacaattcattcgacgcttatatttctagcatctctaactatatcgaagcaggcaaaatcgagagtatgcgcgaattttataacccaattcgtttgaaaaatgcgcataccgaccaaacagtcgaaagtttagcggagcacggcgtggaatatttagagattcgctctattgacctaaatccacttgaaccaaatggcatttctaaagacgagcttgattttattcacctatttttaatcaaaggtttgctttcagaagatcgtgagttgtgcgcaaataatcaacaattggccgatgaaaatgaaaataatattgccctaaatggcctcgcgcagccctcaataaaaaattgcgataatgaagacataccactcgcagatgctgggcttttagaactcgacaagatgagcgacttcatcaaaagtctgcgaccagaagacaccaaactccgagcaatcatcgaaaaacaaaaagaacgtctgctacaccctgaaaaaacgattgctgcacaagtgaaacaacaagtaacaaaagaaggctacgtggacttccatttaaaccaagccaaaacttatatggaagaaaccgaagctctagcctacaaattgattggcgcagaagatatggagctttccacccaaatcatatggaaagacgccattgcgcgtggcatcaaagtcgacgtattagaccgagctgaaaacttccttcgcttccaaaaaggcgaccacattgaatatgtaaaacaagcgagcaaaacgtccaaagataattacgtttccgttttaatgatggaaaacaaagtcgttacaaagcttgtacttgcagaacacgatatccgagtgccctttggcgatagttttagtgaccaagctcttgcccttgaagcattctccctatttgaggataaacaaatcgtcgttaaaccaaaatccaccaactatggttggggaattagtattttcaaaaacaaattcacgctagaagattaccaagaagctttaaatatcgcattcagttatgatagctctgtcattattgaggagttcattcctggcgacgagttccgcttcttagttattaacgacaaagttgaagctgtactaaaacgtgtccccgctaacgtaaccggcgacggaattcatacagtgcgcgagctggtcgaagaaaaaaacacagatcctttgcgcggaacagatcatttgaaaccacttgaaaaaatccgtactggtccagaagaaactctaatgctttccatgcaaaacctttcttgggatagcattcctaaagcagaagaaatcatctaccttcgcgaaaactccaacgtcagcacaggtggcgatagcattgactatactgaagagatggacgattacttcaaagagatagctattcgcgctacacaagtacttgacgccaaaatttgcggcgtagacataattgttccacgtgaaacaattgatcgcgataaacatgctatcattgagctaaacttcaacccagcgatgcacatgcactgcttcccttatcaaggagagcagaaaaaaattggtgataagattttagatttcttatttgactaa

SEQ ID NO: 8 - protein, 776 aa, Listeria monocytogenes
MIKLDMTMLDSFKEDPNLRKLLFSGHFGLEKENIRVTSDGKLALTPHPAIFGPKEDNPYIKTDFSESQIEMITPVTDSIDSVYEWLENLHNIVSLRSENELLWPSSNPPILPAEEDIPIAEYKTPDSPDRKYREHLAKGYGKKIQLLSGIHYNFSFPEALIDGLYANISLPEESKQDFKNRLYLKVAKYFMKNRWLLIYLTGASPVYLADFSKTKHEESLPDGSSALRDGISLRNSNAGYKNKEALFVDYNSFDAYISSISNYIEAGKIESMREFYNPIRLKNAHTDQTVESLAEHGVEYLEIRSIDLNPLEPNGISKDELDFIHLFLIKGLLSEDRELCANNQQLADENENNIALNGLAQPSIKNCDNEDIPLADAGLLELDKMSDFIKSLRPEDTKLRAIIEKQKERLLHPEKTIAAQVKQQVTKEGYVDFHLNQAKTYMEETEALAYKLIGAEDMELSTQIIWKDAIARGIKVDVLDRAENFLRFQKGDHIEYVKQASKTSKDNYVSVLMMENKVVTKLVLAEHDIRVPFGDSFSDQALALEAFSLFEDKQIVVKPKSTNYGWGISIFKNKFTLEDYQEALNIAFSYDSSVIIEEFIPGDEFRFLVINDKVEAVLKRVPANVTGDGIHTVRELVEEKNTDPLRGTDHLKPLEKIRTGPEETLMLSMQNLSWDSIPKAEEIIYLRENSNVSTGGDSIDYTEEMDDYFKEIAIRATQVLDAKICGVDIIVPRETIDRDKHAIIELNFNPAMHMHCFPYQGEQKKIGDKILDFLFD

SEQ ID NO: 9 - DNA, 2331 bp, Listeria innocua
atgataaaacttgatatgacgatgctcgattcttttaaagaaaacgaagctctccggaaatacttattttctggtcattttggtttggaaaaagagaatatacgcgtaacttctgatggaaaattggctcttacgcctcatccagcaatttttggcccaaaagaagataacccatatattaaaaccgatttttcggaaagccaaattgaaatgattacgccagtgaccgactcgattgacgctgtatatgaatggcttgaaaatcttcataatatcgtttcactacgcgcagaaaacgaactgctttggccttctagcaatccgccaattttaccagcagaagaagatattcccattgcagaatacaaaacgcccgacagccctgatagaaaatatcgcgagcacttggctaaaggctacggtaagaaaatccaattattatccggaattcactataatttctcttttcctgaagcgctaattgacgggctttatgccgaaatcagccatcctaacgaatctaaacgcgactttaaaaatcgcttatatttaaaagtagctaaatatttcatgaaaaatcgttggttgcttatttatttaactggcgcgagtccagtttatctcgctgattttacaaaaacaaacagcgaagaagtactgaacgatggtagcaaagcgcttcatcgcgggatttcccttcgcaatagtaatgccggctataaaaacaaagaatcgcttttcgtcgattacaattcatttgacgcttatatttcgagtatttccaactacattgaagctggtaaaatcgaaagtatgcgcgaattttataatccaatccgtttaaaaaatgcccatactgatcaaactgttgaaagtttagctaagcatggtgtggaatatctagaaattcgctcgattgacttaaatccacttgaaccaaacggaatttccaaagatgaacttatttttatccacctatttttaattaaaggtttgctttcggaagatcgtgagctatgcgcgaacaaccaacaactagccgacgaaaacgaaaataatatcgcgttaaacggccttgcacagcccgcaattaaaaactgtgataacgaggaaatgtcccttgctgatgcaggacttttagaattagacaaaatgagcgacttcatccaaagtctaattcctaacgacaaccatttccaagcaatcattgaaaaacaaaaagaacgcctgctacaccctgaaaaaacgattgctgcacaagtacaagcacaatcagctaaagaaggttacgtcgaattccatttaaaccaagcaaaaacttatatggaagaaacagaggcgctggcttataaactcgttggcgcagaagacatggaactttccacacaaatcatttggaaagacgccatcgcgcgcggtatcaaagttgacgtattagaccgggccgaaaacttccttcgcttccaaaaaggcgaccatgtggagtatgtgaaacaagccagcaaaacttctaaagacaactatgtatctgtgttaatgatggaaaataaagttgtcaccaagcttgtactagcagagcacggcattcgcgtaccatttggcgatagttttagcgatcaagcgcttgcacttgaagctttctctttatttgaagataagcaaatcgtcgttaaaccaaaatctaccaactatggttggggtatcagcattttcaaaaacaaatttacgctagaagattaccaagaagctttaaatatcgcattcagttacgatagctccgtcattatcgaggagttcattcctggcgacgagttccgtttcttagtcattaacgacaaagttgaggctgttttaaaacgcgtaccagctaacgtaactggcgacggaatccatacagtgcgccaactggttgaagaaaaaaacaccgatccattgcgcggaacggaccatttaaaaccacttgaaaaaatccgtactggtcctgaagaaactctcatgctatccatgcaaaatctttcttgggatagtattcctaaagcagaagaaatcatctaccttcgcgaaaactccaatgtcagcacaggtggcgacagcattgactataccgaagaaatggatgactacttcaaagaaatagctattcgcgccacccaagtccttgatgccaaaatttgcggcgtagacataattgttccacgtgaaacaattgaccgcgataagcacgctatcatcgagctaaacttcaatccagcgatgcacatgcactgcttcccttatcaaggtgagaagaaaaaaattggtgataagattttagatttcttgtttgaataa

SEQ ID NO: 10 - protein, 776 aa, Listeria innocua
MIKLDMTMLDSFKENEALRKYLFSGHFGLEKENIRVTSDGKLALTPHPAIFGPKEDNPYIKTDFSESQIEMITPVTDSIDAVYEWLENLHNIVSLRAENELLWPSSNPPILPAEEDIPIAEYKTPDSPDRKYREHLAKGYGKKIQLLSGIHYNFSFPEALIDGLYAEISHPNESKRDFKNRLYLKVAKYFMKNRWLLIYLTGASPVYLADFTKTNSEEVLNDGSKALHRGISLRNSNAGYKNKESLFVDYNSFDAYISSISNYIEAGKIESMREFYNPIRLKNAHTDQTVESLAKHGVEYLEIRSIDLNPLEPNGISKDELIFIHLFLIKGLLSEDRELCANNQQLADENENNIALNGLAQPAIKNCDNEEMSLADAGLLELDKMSDFIQSLIPNDNHFQAIIEKQKERLLHPEKTIAAQVQAQSAKEGYVEFHLNQAKTYMEETEALAYKLVGAEDMELSTQIIWKDAIARGIKVDVLDRAENFLRFQKGDHVEYVKQASKTSKDNYVSVLMMENKVVTKLVLAEHGIRVPFGDSFSDQALALEAFSLFEDKQIVVKPKSTNYGWGISIFKNKFTLEDYQEALNIAFSYDSSVIIEEFIPGDEFRFLVINDKVEAVLKRVPANVTGDGIHTVRQLVEEKNTDPLRGTDHLKPLEKIRTGPEETLMLSMQNLSWDSIPKAEEIIYLRENSNVSTGGDSIDYTEEMDDYFKEIAIRATQVLDAKICGVDIIVPRETIDRDKHAIIELNFNPAMHMHCFPYQGEKKKIGDKILDFLFE

SEQ ID NO: 11 - DNA, 2271 bp, Enterococcus faecalis
atgaattatagagaattaatgcaaaagaaaaatgttcgtccttacgtattgatggctcgttttggtttagaaaaagaaaaccaacgtagtacacgagaagggcttttagcgacaactgagcatcccacggtttttggtaaccgttcttatcatccatatattcaaacagattttagtgaaacacaattagaactaatcacgcctgtagcaaatagcggcacagaaatgcttcgttttttagatgccattcacgatgtggctcgtcgttcgattccagaagatgaaatgctgtggccattaagtatgccgccacaattaccaacaaaagatgaagagattaaaattgctaaattagatcaatatgatgcggtgttatatcgtcgttatttggcaaaagagtatggcaaacgaaaacaaatggtcagcggaattcattttaattttgaatatgaccaagccctgattcagcaattatatgatgaacaatccgaagtgacagattgtaaacaatttaaaacgaaagtgtacatgaaagttgcccgtaactttttacgttatcgctggttaattacgtatctttttggggcttcaccagttagtgaagaccgctactttagggtctacgacgaccaaccgcaagaacctgttcgcagtattcggaatagtacgtatggctacagaaatcatgacaatgtgaaagtatcgtatgcctcattggaacgctatttagaagatattcatcgcatggtggaaaatggtttactttctgaagaaaaagaattttatgcgcctgtgcgcttacgtggcgggaaacaaatgtctgatctgcctaaaacaggtattcgctatatcgagttgcgtaatttagacttaaatcctttttcacgtttaggcattgtggaagatactgtggatttcttacattatttcatgttgtatttattgtggacagatgaaaaagaagaagcggatgaatgggtgaaaactggggatatttttaatgaacaagtggctcttggtcatcctcatgaaacgattaagttaattgcagaaggcgatcggattttttcagaaatgattgatatgttagatgctctaggcattcgcaaaggcaaagaagttgtcggtaagtattatcaacaactgcggaatccacaagacaccgtttctggcaaaatgtggacgattattcaagaaaactccaacagtgaactgggaaatatttttggaaaccaatatcaaagtatggcctttgaacgcccttatcaattagctggtttccgtgagatggaattatccacacaaattttcttatttgatgcgattcaaaaaggtttggaaatcgaaattttagatgaacaagaacaatttttgaaactgcaacatggcgagcacattgaatacgtcaaaaatgccaacatgactagcaaagataactacgtggtaccattgattatggaaaacaaaaccgtgacaaagaaaattttgtctgcagcaggtttccatgtgcctggcggtgaagaattttcatcttttattgaggcacaagaagcacatttacgctacgccaataaagcgtttgtcgtgaaaccaaaatcaacgaattacggtttaggaattacgatttttaaagaaggagcttcgttggaagactttacggaagcgttacggattgcttttaaagaggacacagcggttttaattgaagaatttttacctggaacagaatatcggttctttgtgttagataatgatgtaaaagccatcatgttgcgcgtgccagccaatgttaccggagatggcaaacacactgtagaagaattggtggccgctaaaaatagtgatccattgcgggggaccaatcaccgtgcaccactagagttaatccagttaaatgatttagaaaaactaatgttgaaagaacaaggtttaactatctattctgtgccagaaaaagagcaaatcgtgtacttgcgagaaaattctaatgttagcacgggcggggattcgattgatatgaccgatgtcattgatgatagttataaacaaatcgccattgaggccgtagctgctttaggagccaaaatttgtggcattgatttaatcattcctgacaaagacgtaaaaggcacacgtgatagcttaacgtacgggattatcgaagcgaactttaatccagccatgcacatgcatgtgtatccatacgctggacagggtagacgcttgacaatggacgttttaaaacttttatacccagaagtggttcaataa

SEQ ID NO: 12 - protein, 756 aa, Enterococcus faecalis
MNYRELMQKKNVRPYVLMARFGLEKENQRSTREGLLATTEHPTVFGNRSYHPYIQTDFSETQLELITPVANSGTEMLRFLDAIHDVARRSIPEDEMLWPLSMPPQLPTKDEEIKIAKLDQYDAVLYRRYLAKEYGKRKQMVSGIHFNFEYDQALIQQLYDEQSEVTDCKQFKTKVYMKVARNFLRYRWLITYLFGASPVSEDRYFRVYDDQPQEPVRSIRNSTYGYRNHDNVKVSYASLERYLEDIHRMVENGLLSEEKEFYAPVRLRGGKQMSDLPKTGIRYIELRNLDLNPFSRLGIVEDTVDFLHYFMLYLLWTDEKEEADEWVKTGDIFNEQVALGHPHETIKLIAEGDRIFSEMIDMLDALGIRKGKEVVGKYYQQLRNPQDTVSGKMWTIIQENSNSELGNIFGNQYQSMAFERPYQLAGFREMELSTQIFLFDAIQKGLEIEILDEQEQFLKLQHGEHIEYVKNANMTSKDNYVVPLIMENKTVTKKILSAAGFHVPGGEEFSSFIEAQEAHLRYANKAFVVKPKSTNYGLGITIFKEGASLEDFTEALRIAFKEDTAVLIEEFLPGTEYRFFVLDNDVKAIMLRVPANVTGDGKHTVEELVAAKNSDPLRGTNHRAPLELIQLNDLEKLMLKEQGLTIYSVPEKEQIVYLRENSNVSTGGDSIDMTDVIDDSYKQIAIEAVAALGAKICGIDLIIPDKDVKGTRDSLTYGIIEANFNPAMHMHVYPYAGQGRRLTMDVLKLLYPEVVQ

SEQ ID NO: 13 - DNA, 2337 bp, Clostridium perfringens
atggtgaatttagataaaggtttattaaaaattataaaagatgaaagtttagaagattactttataaaggctaattttggtttagaaaaagaaaatgttagggttacagaaagcggaaacttagctttaacacctcatccaaaggcctttggagatagggaaaagaatgcatatataaaaacagatttttctgagagtcagttagaaatggtaacacctgtctgtaatactttagaggaagtttatagttttatatgtaatttaaataaggttgtatctttagagattatgaaaaatggagaatttttatggccacagagtaatcctcctatattaccaagggaagaggagattccaattgctaagctttctaatagagaagatgaattatatagagaaaatcttagttataaatacggcaaaaagaaacaagttataagtggaatacattataatttttcatttaaagaggaatttattaagttactttacaaagagcttaaagtggaaaaggattttagagaatttaaagatgatatctaccttagaatggctagaaactttcaaaaatatcattggttattaatatacttaactggagcaagtcctgttttccatgaaagttatatagaggaaattaaagaagagggtgaaaaattaggagaggattcttattatataaaagatgatacttctttaagaaatagttcttatggatataaaaataaaaaggactattatgtttcatataacagtatagaagaatatgctagtgacataaagaatttagttaaggataaggaaatacaaagtataaaagaatattataatcctataaggttaaaatcactaggaagcgaagatatgcttgaaagcttacttcacaaggggattgattacttagaggtaagacttttagacttagatcctttaagtattcaaggagtgagtaaagaaacactttatctattgcatttatttatgatttatactttattaaaagagaacaaggaaataacatataaagatcaagaagaatttttcaaaaatcatgatatggttgctttaaagggtagaaatgaagaggccgttatatatgaaaatggagttcctgttttattaaaagacaaaggaagagaaatactaagtgaaatggatgaaattgtagagatcttattttcaaataatgaggaatttaaaaatgttatcaaaagagcattagaaaaaattaataatcctcatgatacaatttcagagaaacttattaaggatataaaagaagagggatatattaatttccacatgagattagctaaggagtacttaaataactttaaaaataaagaatttaatttagttggttatgaagatttagaattatcaacacaaatattaatattagatgctattaaaagaggaatagagtttaatattatggatagattagagaactttatttctcttagtgatggagaaaaggttgaatatgttaagcaagcaacaaaaacttctaaggattcatatataactgccttaataatggaaaataaattagttactaaggatattttaagggaaaataatataagggttcctaaggggaaagattatgacaatatagatgaggcaaagaaagattttagattatttaaggatgagaaaatagttataaaacctaagtctactaattttggtttaggaattagtatatttcctggagaatattcaagggaagactatgataaagctgttgaaatagcttttagagaggatagttcaatccttatagaagaatttatgacaggaaaggaatatagatttcttgtaataggggaagaggtagtaggaatacttcatagagaacctgctaatgtaattggtaatggagaaagtaccatagaagagcttgtttctgaaaaaaataaagatccattaagaggaaagggatataagactcctttagaaaaaataaaattaggagagatagaagaaatgtttttgaaaaatcaaggactaagctttaagtctattcctaaaaatggagaaaaaatctatttaagagaaaactctaatataagcacaggaggagacagcatagactttactgacaaaatacatcctagttataaagaggtggcattaaagtctgctaaggctgttaaagcccttatatgcggagtagatatggtaatagataatatagaggaagaggcaaaggaaaaaaatcatggcataatagaattgaattttaacccagcaatacatattcattgtttcccttataaaggagagaatagaaaagctggtgaaaagatattagatttattgtttaattaa

SEQ ID NO: 14 - protein, 778 aa, Clostridium perfringens
MVNLDKGLLKIIKDESLEDYFIKANFGLEKENVRVTESGNLALTPHPKAFGDREKNAYIKTDFSESQLEMVTPVCNTLEEVYSFICNLNKVVSLEIMKNGEFLWPQSNPPILPREEEIPIAKLSNREDELYRENLSYKYGKKKQVISGIHYNFSFKEEFIKLLYKELKVEKDFREFKDDIYLRMARNFQKYHWLLIYLTGASPVFHESYIEEIKEEGEKLGEDSYYIKDDTSLRNSSYGYKNKKDYYVSYNSIEEYASDIKNLVKDKEIQSIKEYYNPIRLKSLGSEDMLESLLHKGIDYLEVRLLDLDPLSIQGVSKETLYLLHLFMIYTLLKENKEITYKDQEEFFKNHDMVALKGRNEEAVIYENGVPVLLKDKGREILSEMDEIVEILFSNNEEFKNVIKRALEKINNPHDTISEKLIKDIKEEGYINFHMRLAKEYLNNFKNKEFNLVGYEDLELSTQILILDAIKRGIEFNIMDRLENFISLSDGEKVEYVKQATKTSKDSYITALIMENKLVTKDILRENNIRVPKGKDYDNIDEAKKDFRLFKDEKIVIKPKSTNFGLGISIFPGEYSREDYDKAVEIAFREDSSILIEEFMTGKEYRFLVIGEEVVGILHREPANVIGNGESTIEELVSEKNKDPLRGKGYKTPLEKIKLGEIEEMFLKNQGLSFKSIPKNGEKIYLRENSNISTGGDSIDFTDKIHPSYKEVALKSAKAVKALICGVDMVIDNIEEEAKEKNHGIIELNFNPAIHIHCFPYKGENRKAGEKILDLLFN

SEQ ID NO: 15 - DNA, 2340 bp, Desulfotalea psychrophila
atggcttttagcaaaaacatcctcgacagcctcccacctctcatctcaaaacagatcttcgaaggtttttttggttttgaaaaggaaaatatccgggtagacagcaggggtaaacttgccctcacccctcaccccagagaacttggggaaaagacgagccacccctatatcaccaccgatttttccgaaagccagattgaaataataaccccgcccctaccctccatcgccgaatccctaggttttctggagaccctgcacgacctggtcagcatagagctcaaggacgaatatctctggccccagagtgccccgcccatattaccggaaagagaagaagatatccccattgcccattttgggggcgagtttcgggaacaagaagagtaccgtctgcaactggccaagatatatggacgaaaacggcagatgttttcaggtatccacttcaacatctcccttcccgaaagatttctggaactgctccatgaagagggaaagcaagaacagccctttgcagagtttagagaagatatctatatgaaaaccgtgcgtaattttctccgtcatcgctggtttctcatctgcctgttgggggcaagcccggttatccataagtcctaccgtaaacactgcatagatatgctcagtccattcgccaaggatgcctaccatttcccctatgccacctccatacgaaacaatatctgtggctacagaaacacccaggattttcacctgaactacagcaccctgacagattacagggaaagtctgcaagagttggtggagaaaaaagttctccgggatatacgggaaaattacgccccgatacggatcaaaaccaccaccgaccctaaaagaatcaatcacctggagatccgcctgcttgatctcaacccctttttcaagacaggagtcaatcctctacatgcagagataattcacattttcctcatctactgcctactctgccccgaagagacgtcctttaccagcaaagagcaagaaactgccaatagaaaccaggaacaggccgcaactgaaggccttaatccaggtgctatcatctgcgacgctgatggcaacgagcagaggctggacaaacagcttgcccattgcttgcaagagatccagcaaacagtaagcccacacctcccgccggaatacagggcagggatggaagagctggagaggctggtgcaaaaccaagcgtctcgccccaccgatacactgctcaaagagatcaagcaggaggggtttaccgaatggcatatgaaacaggccttaaagttccttaaaaaaagtcacgatgaacagtttatcttccacggtctgagagatatggaactctccacccaactcctccttaggcgggcagccctgcgcggagtatcctttgagatcatggatcggcaggaaaactttgtctgcctggaacaggcgggtaagagagaatatgtaatgcaggccagccgcacctcactggacaattacatcagcgttctcagcatggaaaacaaggtcatcaccaaaaaaatactcgaccaagcgggaatcaacaccccaaagggaagaagctatagcagcccctcggaagcgctggcagactatccctactaccggggcagggctatcgtcatcaaaccgaaatccaccaactttggcattggcataacaatcataaaggagaacaacagacatgatttttttgcgcagggtatcgctcaggccttcaagcatgaagcgacggttcttatcgaaaacttcagcagcggaaaagaatatcgtttctttatcgtcaacgaccaagtcgttggaattctgcacagagtacctgccaatgtcacgggcgacggaacatcaagcgtacaggtactagtgacagaaaagaacaagaaccccctgcggggcagagggtatcgaaccccgctcgagaagatcaaactggaggaaactgaagagatgtttctcgctagccaaggctactcctttgccaccgttccggccaaggatcagcgcatctatctgcgggaaaactcaaatatcagcacaggcggagacagcatagacttcaccgacaaggtgccccaatcatacaaggatattgccgtgcgagccgcccaagctctgcaggtaaaaatcaccggtctcgatatgatgattgactctctagaagaggatgccgctgaagataatttcagtattatcgagctgaacttcaaccctgccatccatatccactgccatccctatattggcaagaacagacatctcgacgacaagatactggatgccctcggcttcacaggagctgaagaggctggggagaaggcatag

SEQ ID NO: 16 - protein, 779 aa, Desulfotalea psychrophila
MAFSKNILDSLPPLISKQIFEGFFGFEKENIRVDSRGKLALTPHPRELGEKTSHPYITTDFSESQIEIITPPLPSIAESLGFLETLHDLVSIELKDEYLWPQSAPPILPEREEDIPIAHFGGEFREQEEYRLQLAKIYGRKRQMFSGIHFNISLPERFLELLHEEGKQEQPFAEFREDIYMKTVRNFLRHRWFLICLLGASPVIHKSYRKHCIDMLSPFAKDAYHFPYATSIRNNICGYRNTQDFHLNYSTLTDYRESLQELVEKKVLRDIRENYAPIRIKTTTDPKRINHLEIRLLDLNPFFKTGVNPLHAEIIHIFLIYCLLCPEETSFTSKEQETANRNQEQAATEGLNPGAIICDADGNEQRLDKQLAHCLQEIQQTVSPHLPPEYRAGMEELERLVQNQASRPTDTLLKEIKQEGFTEWHMKQALKFLKKSHDEQFIFHGLRDMELSTQLLLRRAALRGVSFEIMDRQENFVCLEQAGKREYVMQASRTSLDNYISVLSMENKVITKKILDQAGINTPKGRSYSSPSEALADYPYYRGRAIVIKPKSTNFGIGITIIKENNRHDFFAQGIAQAFKHEATVLIENFSSGKEYRFFIVNDQVVGILHRVPANVTGDGTSSVQVLVTEKNKNPLRGRGYRTPLEKIKLEETEEMFLASQGYSFATVPAKDQRIYLRENSNISTGGDSIDFTDKVPQSYKDIAVRAAQALQVKITGLDMMIDSLEEDAAEDNFSIIELNFNPAIHIHCHPYIGKNRHLDDKILDALGFTGAEEAGEKA

SEQ ID NO: 17 - DNA, 2268 bp, Enterococcus faecium
atgatgaattttaagcaattattattgcatgtcaatgcgcgcccttttatcgaccaagctcgatttggtatagaacgggaaggacagcgggttgatcttgcaggaaatctagcaaaaaccgatcatccagcaatctttggcgatcgatcctatcacccctatatccaaacagattttagcgaaacacaaacagagatgatcacccctgttaccgattctattcccgaattatttcagtatctcgctgctgtttatgatgtgactgctcgttctataccgaaagaagagatgatctggccattaagtatgccacctgccttaccggaaaaagacgaagagatcattattgcaaaattaaaaaatttcgaagatgtcttgtatcgacgttatttagcaaaagaatacgggaagcgaaaacagatggtaagcggtattcatttcaattttgaatttggcgatgagttgctaagaacattgttcagccatcaggaagaatttcaagatttttccgaattcaaaacagaactttatttgaaaacagcgagaaactttatgcgctatcgctggatgatcacttatttattcggtgcttcaccaatgagtgagaagaactactttttagatgaatcacatccgcaagaacctgttcgcagtattcgcaacagtgcgctgggctatacgaatcatccaaatgtgaaagtttcctatgcttctatgaaacagtatttagcagatatcgagcgaatgatcgaagaagggaaactttcggaggagaaagaattttatacaccgcttcgtttccgaggcggaaagaaagtcgcagatttagcaacaacaggtgttcgttatattgaattgcggaatatcgatttgaatccttatgcaagattgggtatcaatccagagcaagttcggttcttacaattattcctgatgtacatgttatggacagaagaaaaagaagactgtgaccaatgggtagcagaaggtacgactcgaaataacaaagtagcactagaacaaccatcagatcaaacagaatttcatcaagaaggtagagaaattcttgaaggaatgaagcaaatgctggttgaattggattggttggattctctttatttagtagaggaagcattgactcaaatggatcatcctgaacaaacgttagcagcgaaactctatcaagaagcacagctatcgagtcagcaagaagttgctgtggcattgggacatcaatattataaagaaagccatgaacgtccctatcagttggccggttttcgcgaaatggaactttctactcagatatttatgttcgatgccatccaaaaaggtgtccaagtcaaagtattagatgagtcggatcaatttttgcgtctgcaattccaagaccatgtagaatatgtgaaaaatgcaaacatgacaagtaaggacagctatatcgtcccacttatcatggaaaacaaaacagtaacgaaaaaagtcttaaaagaagctgggttccgggtaccaggaggcgcagaattttcatccatggaagaagcggtaaaagcttatccgagatttgccgagcaggcatttgtgatcaaacctaaatcaaccaattacgggttaggtatcacgattttcaaggaaggagctagtctggaagattatcaagcaggtttagccattgcttttcgtgaagacagttcggttttagtagaagaatttatgccgggaacggaatatcgtttctttgtgatcgacggagaagtccaagccatcatgttgcgagtccctgctaatgttattggtgacagtatccgaacagtgaaagaacttgttgaagaaaaaaatagtgaccctttgcgtggaacgaatcatcgtgcacctttagaattgatccaattaggtgaattagaacaattgatgttgaaagaacaagggttgacgatcgaatcagttccccaagccaatcaaattgtttatctaagggagaactcaaatatcagtacaggtggtgattcgatcgatatgactgatgaattttctgaagcttacaaaaaaatcgccgtttctgcggtagaagcactaggagctaagattagtggtatcgatttgattataccagataaagaaatcgatcctacaactgataaaaaagcatatgggatcatcgaagcaaatttcaatccagccatgcatatgcatgtttatccttttgcaggaaaaggcagaagattgacgatgaatgtattgaaattgttatatcctgaagtattttaa

SEQ ID NO: 18 - protein, 755 aa, Enterococcus faecium
MMNFKQLLLHVNARPFIDQARFGIEREGQRVDLAGNLAKTDHPAIFGDRSYHPYIQTDFSETQTEMITPVTDSIPELFQYLAAVYDVTARSIPKEEMIWPLSMPPALPEKDEEIIIAKLKNFEDVLYRRYLAKEYGKRKQMVSGIHFNFEFGDELLRTLFSHQEEFQDFSEFKTELYLKTARNFMRYRWMITYLFGASPMSEKNYFLDESHPQEPVRSIRNSALGYTNHPNVKVSYASMKQYLADIERMIEEGKLSEEKEFYTPLRFRGGKKVADLATTGVRYIELRNIDLNPYARLGINPEQVRFLQLFLMYMLWTEEKEDCDQWVAEGTTRNNKVALEQPSDQTEFHQEGREILEGMKQMLVELDWLDSLYLVEEALTQMDHPEQTLAAKLYQEAQLSSQQEVAVALGHQYYKESHERPYQLAGFREMELSTQIFMFDAIQKGVQVKVLDESDQFLRLQFQDHVEYVKNANMTSKDSYIVPLIMENKTVTKKVLKEAGFRVPGGAEFSSMEEAVKAYPRFAEQAFVIKPKSTNYGLGITIFKEGASLEDYQAGLAIAFREDSSVLVEEFMPGTEYRFFVIDGEVQAIMLRVPANVIGDSIRTVKELVEEKNSDPLRGTNHRAPLELIQLGELEQLMLKEQGLTIESVPQANQIVYLRENSNISTGGDSIDMTDEFSEAYKKIAVSAVEALGAKISGIDLIIPDKEIDPTTDKKAYGIIEANFNPAMHMHVYPFAGKGRRLTMNVLKLLYPEVF

SEQ ID NO: 19 - DNA, 2298 bp, Mannheimia succiniproducens
atgcggtttgatcaaggaaatcttatgaatattcaacaaattgttaaagaaaagggattgggtttacttttccggcaaggtactgtgggcattgaaaaagagagccaacgcgtacatgccgacgggtcgattgttaccagcgaacacccgaaagcctttggtaaccgttcctatcatccttatattcaaacagattttgccgaaagccagttagaattaattacgccgccgaataaaaaaatcgaagatacgttacgttggctgtccgccctgcatgaagtgacgttgcgtactatcgacgaaaacgaatatattttcccaatgagtatgcccgcgggattgccgccggaacaggaaatccgggtcgcccaattggataatgcggcggatgtggcttatcgcgagcatttggttgcctcttacggcaaggccaagcaaatggtaagcggcattcattataattttcaattggatccaaaactggttgaaacgctgtttaacgcacaaacggactataaaagtgcggtcgattttcagaataatttatatttgaaaatggcaaaaaatttcctgcgttatcaatggataccgctttatttattatcagctacgccaaccgtggaggcgaattatttcaaagacggatcaccgttaaaacccaatcaatatgtgcgcagtttgcgttccagtaaatacggttatgtgaatgcgccggatattatcgtttcttttgacagtattgaaaaatatgtggaaaccttggaacattgggttaattccggcagattaatcgctgaaaaagaattttattccaatgtccgcctgcgcggggcgaaaaaagcccgcgaatttttgcatacggggattcaatatcttgaattccgcttgtttgatctgaacccttttgaggcttacggcattaatttaaaggacgcaaaatttattcatcattttattttgttaatgatttggctggaggaaacggcggatcaagatgccgttgagttagggcgtgccagattaggcgaagtggcgtttgaagaccctcacagcgaaaccgcttatcgtgatgaaggcgaacagattattaatcaattgattgatatgctgaaagctataggcgcggaacaaagcgcggtggaattcgccgaagaaaaattagcccagtttgccaatccgggtcaaactctttgcgcccgtttagtcgatgccattgaacaggccggcggttatcaacaattgggcggggaaatcgcaaaacgtaataaagttcaggcttttgagcgtttttatgctttatccgcctttgataacatggagctttctactcaggctttaatgtttgatgccattcaaaaaggcttaaatatggaaattcttgatgaaaacgaccagttcctgcgcctgcaattcggtgatcattttgaatacgtgaaaaacggcaatatgacctcgcatgacagttatatttcgccgttaattatggaaaataaagttgtcactaaaaaagtactggcgaaagcggggttcaatgtgccgcagagtttggaatttaccagtgtcgagcaagcggtggcaagctatccgttatttgaaggcaaagcggttgttatcaagccgaaatccactaactttggtttgggaatcagtatttttcaacaaggcgtgcatgacaaggcggatttcgccaaagccgttgaaattgcgttccgtgaagacaaagaagtgatggtggaagattatctggtgggtacggaataccgtttcttcgtactgggtaacgaaacccttgcggtattattgcgcgtgccggcgaatgtgatgggcgatggtgtccatacggtggcggaacttgtggcggcgaaaaatgatcatccgttgcgcggtgacggcagtcgtacgcctctgaagaaaatcgccttaggtgagattgaacaacttcaattaaaagagcagggattaacggtagattccgtgccggcaaaagatcagctcgtacagttacgggccaattcgaatatcagtaccggcggcgatagtatcgatatgaccgatgaaatgcaccctagctataaagatttagcggtaggcattactaaggcaatgggggcggcggtgtgcggcgtggatttaattatccctgatttgaaaaaaccggcggagccgaatttaagctcatggggcgtaattgaagcaaactttaatccgatgatgatgatgcatatttttccgtattccggtaaatcaagaagattgacgttgaatgtgttggggatgttgtttccggaattagtctag

SEQ ID NO: 20 - protein, 757 aa, Mannheimia succiniproducens
MNIQQIVKEKGLGLLFRQGTVGIEKESQRVHADGSIVTSEHPKAFGNRSYHPYIQTDFAESQLELITPPNKKIEDTLRWLSALHEVTLRTIDENEYIFPMSMPAGLPPEQEIRVAQLDNAADVAYREHLVASYGKAKQMVSGIHYNFQLDPKLVETLFNAQTDYKSAVDFQNNLYLKMAKNFLRYQWIPLYLLSATPTVEANYFKDGSPLKPNQYVRSLRSSKYGYVNAPDIIVSFDSIEKYVETLEHWVNSGRLIAEKEFYSNVRLRGAKKAREFLHTGIQYLEFRLFDLNPFEAYGINLKDAKFIHHFILLMIWLEETADQDAVELGRARLGEVAFEDPHSETAYRDEGEQIINQLIDMLKAIGAEQSAVEFAEEKLAQFANPGQTLCARLVDAIEQAGGYQQLGGEIAKRNKVQAFERFYALSAFDNMELSTQALMFDAIQKGLNMEILDENDQFLRLQFGDHFEYVKNGNMTSHDSYISPLIMENKVVTKKVLAKAGFNVPQSLEFTSVEQAVASYPLFEGKAVVIKPKSTNFGLGISIFQQGVHDKADFAKAVEIAFREDKEVMVEDYLVGTEYRFFVLGNETLAVLLRVPANVMGDGVHTVAELVAAKNDHPLRGDGSRTPLKKIALGEIEQLQLKEQGLTVDSVPAKDQLVQLRANSNISTGGDSIDMTDEMHPSYKDLAVGITKAMGAAVCGVDLIIPDLKKPAEPNLSSWGVIEANFNPMMMMHIFPYSGKSRRLTLNVLGMLFPELV

SEQ ID NO: 21 - DNA, 2274 bp, Haemophilus somnus
atgaaaatccaacatttaattaagcaacatcaacttggcttattgtttcaacaaggctcctttggtttagaaaaagaaagccaacgagtttatcaagacggttcggtagtaacaaccgaacaccctaaatgttttggtaacagatcttaccatccctacattcaaactgattttgcggaaagtcagctcgaattgatcacgcctcccaataaaaatttagaagatagtttaagatggttatccgctattcatgaagttgttttgcgttctttgcctgaggatgaatttatttttccgttgagtatgccggcaggattgccttcagatgaattgataaaagtcgctcaattggataaccctgaagatgtggcatatcgtgaacatttagttcagtcttatggaaaaaataaacaaatggtcagtggaatccattataactttcagttggctccggaactcattcaaaccttatttgatttacagcaagagcaaaaaagtgcggtagattttcagaataatttatacttgaaaatggcgaaaaattttcttcgttatcaatgggttttattgtacttattatcagcaacaccaacggtagagagtaattattttaaaggtagttcgccattaggaaaaggtgaatatgttcgcagtttacgttcaagcaaatatggttatgtgaatgatcctgaggtcattgtttcttttgatagtttagaacaatatgcagaaagtctggagcattgggttaaaagcggaaaactgatagcagagaaagaattttattccaatgtgcgtttacgaggtgcgaaaaaggcgagagatttaattcaaaatggaattaaatatttagaatttcgcttgtttgatctcaatcctttcgagcaatacggtatgagccttgcggatgcaagatttattcatcattttgtactattgatgatttggcttgatgaaatgccagatcaacaaggtgttgaactggggcgtagccgtctagcggaagtagctctagaaaatccgttggcacaaactgcctatcgagaagaaggtgagtggttacttaatcagttaatcagtatgttacaaagtatcggggcagaccaaagtgcggtgagttttgtccgagaaaaattagctcaatttgctgatcccgggttgacgttatgtggtcgtttagtccaagaaattgagcagaaaggcggttatcaaaaattaggtgcagagcttgccaaacaatataaagaaaatgcttttgagcgtttttatgcgttaaccgcttttgataacatggaactttcaactcaagccttaatgtttgatttaattcagcaaggtatcagttttgagatcttggatgaacgagatcaatttttacgcttacaatttggtgaacatgttgaatatgtaaaaaatggtaatatgacttcgaaagatagctatatttctcctcttattatggaaaataaagtggttacgaaaaaagtattggaaaaggcggggtttaatgttccacagagtcaggagtttacttcagttaaacaagcgatagtcggttatagcctgtttgaaaaaagagcggtcgttattaaacctaaatctactaactatggcttggggattacaatttttcagcaaggtgtgaataatagagaagattttgccaaggcagtggaaattgcgttccgtgaagataaagaaattatggtggaggattatttagttggtactgaataccgcttctttgtgttgggtgaggaaacattggcagttttattacgtgttccggcaaatgtagtaggcgatggcattcatactgtggctgaattggtagctcaaaaaaatgctcactcattgcgtggtgatggtagtcgtacgccattgaaaaaaattgcattaggcgatattgagcaattgcagttgaaagagcaaggtttgactgttgagagtattccagctaaagatcaaattgttcagttgcgggcaaattctaatattagtacaggcggtgacagcattgatataactgatgaaatgcatttgagttataaacaactggcggtggggattgcaaaagctatgggggctgctgtgtgcggtgtggatctgattatttccgatttgaaagaaccggcacagcctgatttaagttcttggggcgtaattgaagcgaattttaatccaatgatgatgatgcacatcttcccttatgccggacaatctagaagattaacaagaaatgtgattaacatgttgtttccggaagtaaagtaa

SEQ ID NO: 22 - protein, 757 aa, Haemophilus somnus
MKIQHLIKQHQLGLLFQQGSFGLEKESQRVYQDGSVVTTEHPKCFGNRSYHPYIQTDFAESQLELITPPNKNLEDSLRWLSAIHEVVLRSLPEDEFIFPLSMPAGLPSDELIKVAQLDNPEDVAYREHLVQSYGKNKQMVSGIHYNFQLAPELIQTLFDLQQEQKSAVDFQNNLYLKMAKNFLRYQWVLLYLLSATPTVESNYFKGSSPLGKGEYVRSLRSSKYGYVNDPEVIVSFDSLEQYAESLEHWVKSGKLIAEKEFYSNVRLRGAKKARDLIQNGIKYLEFRLFDLNPFEQYGMSLADARFIHHFVLLMIWLDEMPDQQGVELGRSRLAEVALENPLAQTAYREEGEWLLNQLISMLQSIGADQSAVSFVREKLAQFADPGLTLCGRLVQEIEQKGGYQKLGAELAKQYKENAFERFYALTAFDNMELSTQALMFDLIQQGISFEILDERDQFLRLQFGEHVEYVKNGNMTSKDSYISPLIMENKVVTKKVLEKAGFNVPQSQEFTSVKQAIVGYSLFEKRAVVIKPKSTNYGLGITIFQQGVNNREDFAKAVEIAFREDKEIMVEDYLVGTEYRFFVLGEETLAVLLRVPANVVGDGIHTVAELVAQKNAHSLRGDGSRTPLKKIALGDIEQLQLKEQGLTVESIPAKDQIVQLRANSNISTGGDSIDITDEMHLSYKQLAVGIAKAMGAAVCGVDLIISDLKEPAQPDLSSWGVIEANFNPMMMMHIFPYAGQSRRLTRNVINMLFPEVK

SEQ ID NO: 23 - DNA, 2265 bp, Streptococcus thermophilus
atgacattaaaccaacttcttcaaaaactggaagctaccagccctattctccaagctaattttggaatcgagcgcgagagtctacgtgtcgataggcaaggacaactggtgcatacacctcacccatcctgtctaggagctcgtagtttccacccctatattcagactgatttttgcgagtttcagatggaactcatcacaccagttgccaaatctactactgaggctcgccgatttctgggagctattactgatgtagctggccgctctattgctacagacgaggttctctggcctttgtccatgccacctcgtctaaaggcagaggagattcaagttgctcaactggaaaatgacttcgaacgccattatcgtaactatttggctgaaaaatatggaactaaactacaagctatctcaggtatccactataatatggaactgggtaaagatttagttgaggccttgttccaagaaagtgatcagaccgatatgattgccttcaaaaacgccctctatcttaagctagctcagaactacttgcgctaccgttgggtgattacctatctctttggggcctcacccatcgccgaacaaggtttctttgaccaggaagttccagaacctatgcgttccttccgtaacagtgaccacggctatgtcaataaggaagagattcaagtatcctttgtaagtctagaagattatgtctcagccattgaaacctatatcgaacaaggagatttgattgcagagaaagaattttactcagctgttcgtttccgtggacaaaaggttaatcgttccttccttgacaagggaatcacctacctagagttccgtaatttcgaccttaacccttttgagcgtatcggtattagtcagactactatggacactgtgcacttactcattttagccttcctttggcttgatagccctgaaaatgtcgaccaagctcttgcacaaggccacgcgctaaatgagaaaattgccctctctcatcctctagaacctctaccttcggaggctaaaactcaggacattgtaactgccctagaccaactggtgcaacactttggacttggtgactatcatcaagatctggttaaacaagttaaggcagcctttgcggatccaaatcaaacgctctctgcccagctcttaccctatatcaaagacaaatctctagccgaatttgctttaaacaaggctcttgcctatcatgattacgactggactgcccactatgctctcaagggctatgaagagatggaactctccacccagatgttgctctttgatgccatccaaaaggggattcactttgaaatattggatgagcaagatcaattcctaaaactttggcaccaagaccatgttgaatacgtcaaaaacggtaacatgacctcaaaagacaactacgtggttccccttgctatggctaataagaccgtaaccaagaagattctagcagatgctagctttccagttccttcaggagacgaatttaccagtcttgaggaaggacttgcctactaccctcttatcaaggataagcaaattgttgtcaaacccaagtcaactaactttggtctgggaatttccattttccaagaacctgccagtcttgacaactatcaaaaagcccttgaaattgctttcgcagaagatacttctgtccttgttgaagaatttattccaggaaccgaataccgtttcttcatcttggatgggcgttgtgaggctgtgcttctgcgtgtcgctgccaatgttattggtgatggcaaacacaccattcgtgaactagtcgctcagaaaaatgctaatccattgcgtggccgtgatcaccggtcacctctggaaatcattgagctaggagacatcgaacaactaatgttagctcaacagggttatacacctgatgatattctcccagaaggaaaaaaggtcaatctgcgtcgtaattccaacatctctacaggtggtgactctattgatgtcactgagaccatggattcctcttaccaagaattagccgcagccatggcaactagcatgggcgcctgggcttgcggggttgatctgataattccagatgaaactcaaattgccaccaaggaaaatcctcattgcacctgcattgagctcaactttaacccttcgatgtatatgcacacctactgtgctgagggtcctggccaagctatcactactaaaatcctagataaactttttccagaaatagtggctggtcaaacttaa

SEQ ID NO: 24 - protein, 754 aa, Streptococcus thermophilus
MTLNQLLQKLEATSPILQANFGIERESLRVDRQGQLVHTPHPSCLGARSFHPYIQTDFCEFQMELITPVAKSTTEARRFLGAITDVAGRSIATDEVLWPLSMPPRLKAEEIQVAQLENDFERHYRNYLAEKYGTKLQAISGIHYNMELGKDLVEALFQESDQTDMIAFKNALYLKLAQNYLRYRWVITYLFGASPIAEQGFFDQEVPEPMRSFRNSDHGYVNKEEIQVSFVSLEDYVSAIETYIEQGDLIAEKEFYSAVRFRGQKVNRSFLDKGITYLEFRNFDLNPFERIGISQTTMDTVHLLILAFLWLDSPENVDQALAQGHALNEKIALSHPLEPLPSEAKTQDIVTALDQLVQHFGLGDYHQDLVKQVKAAFADPNQTLSAQLLPYIKDKSLAEFALNKALAYHDYDWTAHYALKGYEEMELSTQMLLFDAIQKGIHFEILDEQDQFLKLWHQDHVEYVKNGNMTSKDNYVVPLAMANKTVTKKILADASFPVPSGDEFTSLEEGLAYYPLIKDKQIVVKPKSTNFGLGISIFQEPASLDNYQKALEIAFAEDTSVLVEEFIPGTEYRFFILDGRCEAVLLRVAANVIGDGKHTIRELVAQKNANPLRGRDHRSPLEIIELGDIEQLMLAQQGYTPDDILPEGKKVNLRRNSNISTGGDSIDVTETMDSSYQELAAAMATSMGAWACGVDLIIPDETQIATKENPHCTCIELNFNPSMYMHTYCAEGPGQAITTKILDKLFPEIVAGQT

SEQ ID NO: 25 - DNA, 2232 bp, Streptococcus suis
atgttacaaaaattatcaccaaacagccccattcttcaggctacttttggaattgagcgcgaatcacttcgcatcaattcaaaccacagagttgcacaaactccccatcctcataaactgggttctcgtagcttccacccctatatccagacagactacagcgagccacagttagagttgattacaccaatcgctcagtcgactaaggaagcgcgtcgcctactgggagccattacagatgttgctgcacgttctatggataagcaagaatacctctggcccctatccatgccacctgtgatttcagaagaagaaatccaaattgctcaactagatagcgactatgagtaccaatatcgtgtcggtttgggggaacgctatggcaagctcgtccagtctatgtctggcatccattataattttgaattaggaaaagacttaacccaacaactttttgaattaagcgaggaaacagattttatcgcattcaaaaataccctctacctcaaattagcacaaaacttcctcaactatcgttggctcttaacctacctatatggagcaagtagcctagccgaaaaaggattcctaaccacggaagtcggttgcgtccgttccattcgtaattccaagtacggctatgtcaactccgataatgttcatatttccttttcttctctccaacagtacgtggccgacatcgaacaagctgtccaatctggtcaactatccgctgagaaggaattttactcttccgtccgactacgtggagcaaagactagccgtgactacctcagcaaggggatttcttatttagaattccggtccttcgacctgaacccctatgacccgcttgctattagtcaagaaacccttgatacagtccacctctttatcttatctcttctttggctagaccaactgacagatgttgataacaccctagcaaaagcagataaacttaacaatctcatcgccctcagtcatccccacacacctctacctaacgatgccgatgccacgccaatcttgacggccatgaaagccatagttctacactttggactggatgactactatggccaactcattgcacatgcggaagcagcccttcaagaccctcgtctgaccctttctggaaaaatagcagagcaggttgaagatggatctctagaaaagtttggtcagcaacagggacaagtctttcatgactatgcttggacagccccttacgctctcaagggctatgaaaacatggaactctccacccagatgattctctttgacgccattcagctgggcttgaatgtggagattttagatgaagaagaccagtttctcaaactctggcatggtgaccatgtcgagtacatcaaaaatggcaacatgacctccaaggacaattacgtcattcctctcgccatggccaacaaggtcgtgaccaagaaaatcttggaccaggctggttttccagtacctgcaggagctgaatttgcaaacaagacagatgccctgcggtattacggacaggtaaccagctctgctattgtagtcaaaccaaaatctaccaacttcggccttggcatctctatcttccaggagccagccagccttgcagactatgaaaaagccctggacatcgccttttcggaagatagccatgtactagtggaggaattcgtggcgggaaccgagtaccgtttcttcatccttgatggcaagtgcgaggctgttctgctccgcgtagcggccaatgtcgtgggggacggtagctcaaccatccgtgaattagtagagcaaaaaaaccaagatccactgcgagggcgtgaccaccgttcaccgctggagatcattaacttgggcgatatcgaactgcttatgttagaacagcagggctacacacctgatacggtcttgccagaaggcgtccaagcctttctgcgtggcaattccaacatctccaccggtggcgactccattgatatgaccgaccagatggacgagtcctacaagcaactggctgccgctatggcgactgctatgggagcttgggcctgcggtgtagatctgattatccccgaccgcaccaagccagccagcaaggaagatccaaactgcacctgcatcgaactcaacttcaaccctgccatgtacctgcacacctatacttacgcaggacctggacagagcattacgccgaagattttgaaaaaattatttccagagctatag

SEQ ID NO: 26 - protein, 743 aa, Streptococcus suis
MLQKLSPNSPILQATFGIERESLRINSNHRVAQTPHPHKLGSRSFHPYIQTDYSEPQLELITPIAQSTKEARRLLGAITDVAARSMDKQEYLWPLSMPPVISEEEIQIAQLDSDYEYQYRVGLGERYGKLVQSMSGIHYNFELGKDLTQQLFELSEETDFIAFKNTLYLKLAQNFLNYRWLLTYLYGASSLAEKGFLTTEVGCVRSIRNSKYGYVNSDNVHISFSSLQQYVADIEQAVQSGQLSAEKEFYSSVRLRGAKTSRDYLSKGISYLEFRSFDLNPYDPLAISQETLDTVHLFILSLLWLDQLTDVDNTLAKADKLNNLIALSHPHTPLPNDADATPILTAMKAIVLHFGLDDYYGQLIAHAEAALQDPRLTLSGKIAEQVEDGSLEKFGQQQGQVFHDYAWTAPYALKGYENMELSTQMILFDAIQLGLNVEILDEEDQFLKLWHGDHVEYIKNGNMTSKDNYVIPLAMANKVVTKKILDQAGFPVPAGAEFANKTDALRYYGQVTSSAIVVKPKSTNFGLGISIFQEPASLADYEKALDIAFSEDSHVLVEEFVAGTEYRFFILDGKCEAVLLRVAANVVGDGSSTIRELVEQKNQDPLRGRDHRSPLEIINLGDIELLMLEQQGYTPDTVLPEGVQAFLRGNSNISTGGDSIDMTDQMDESYKQLAAAMATAMGAWACGVDLIIPDRTKPASKEDPNCTCIELNFNPAMYLHTYTYAGPGQSITPKILKKLFPEL

SEQ ID NO: 27 - DNA, 2256 bp, Lactobacillus plantarum
atggaattagatgccgttggtaaggcaattgtacagtatcacttagtcccactcgttcatcaggctaatttaggactagaggtcaccatgcaccgggtggacgcccatggtcacttagcgacgacagcacacccgcaagcgtttggatcagcgcaacaaaatcatcagttacgtccgggcttttccgcaagtgctttaaagtttactacgccggtgcgtcgtgacattcctgcattgatggcgtatctgaagggcttgaataccgcagcacggcggtcactcgatgcggacgaacgactttggccactgtcgagtacgcctgtgttgccggatgatctaacgaacgtaccactggctgatgttgatcaagtcagctatcagcgtcgtcgcgacttagctcgtaagtatgagttacagcgattaatgacgactggtagtcacgtgaatatgagcttgaatgaagctttattcacccgtttatatactgagactttccatcagcagtatcacagttatgttgactttcgcaatgcaatttatctgaaagtcgctcagggattggtgcgcatgaactggctgattcagtatttatttggcgcttcaccacgcctagccgttacggatactacgagtcgtccacagcgcagtagtgttcaacatcccgatggtcgctacagtcaagtgacgggagactatacgtcaattgatcgctacgtggccaagttgacggcggctgttcgtcaacagcagttgttgtctgtcaatgattttgacgggccagttcggcttcggagtaatgggcagctagctatgatggcccggcagggggtctattatcttgaataccggggcttggatctcgatccaactagtccagtcggggtggacgcgaacgcggtggcatttgttcgtttgttggcgagttatttcgtaatgatgccggcacttccagctaagatggtatcccaagtcaacgctcaagctgaccaattgacccgtcaagttttgggtgaaaatccaacgacggctagtgctcaggccgtgccggctgttcaagttttagatgcacttgctgattttgttaaaacctatggcctaccaaatgaagatgccgtgttactcaaacagttgaagtcgtgggtcactgatccaaagaagacgctgagtgcgcagattgccatgcaagccgatccgttagcatgggcactcgaacgggctgcacgctatcaggaatcgagcaatgaacgtccgtttgaacttgcgggctttaccgcgctagatctatcgagccagcaactagcccagcaggccttgacgcggggagtgcaggtggacgttgttgacccacacgctaacattttacgattgactaagttaggacggtcgcaattagttgtgaatgggagcggaacggatttaaatccacaggcgctaacgaccgtactgacacataaagcagcggccaaacaaattctggctgagcacggggttccggtgccggcttcacagacatatcatacagctaatcagttgattgctgattatgatcggtacgttcaagctggtgggatcgtattaaaagcggcggatgagtcgcacaaagtaattgtctttcggattatgcccgaacgcggactgtttgaacaagtcgtccggcaactattcgagcaaacgtccgcggtaatggccgaggaagtggtagtcgcatcaagttatcgctttttggttatcgatagtcgtgtgcaagcaatcgtcgaacgaattccagccaatattgttggtgatggtcgctcaacggtcaagacgttacttgatcgcaaaaatggtcgagcgttgcgcgggaccgcttttaagtggcctcaatcagcgctacagttaggaacgatcgaacggtatcgcctggactcatatcacttgaccttagattctgtggtcagccggggaactcagatcttattacgagaggatgcgacttttggtaacggggcggacgtgctagacgcgacggctgatatgcatcaatcctatgtgcaggcggtggaaaagttggtagcagacttacacttggcggtcgctggggtcgacgtgatgattcccaatctctatgccgaattagtgccagagcatcctgaaatggcggtatacttgggtattcatgcggcgccgtacttgtatccgcacttgttcccaatgtttggtactgcccaaccagtggcggggcagttgttggatgcattgtttaaaaatgaagattaa

SEQ ID NO: 28 - protein, 751 aa, Lactobacillus plantarum
MELDAVGKAIVQYHLVPLVHQANLGLEVTMHRVDAHGHLATTAHPQAFGSAQQNHQLRPGFSASALKFTTPVRRDIPALMAYLKGLNTAARRSLDADERLWPLSSTPVLPDDLTNVPLADVDQVSYQRRRDLARKYELQRLMTTGSHVNMSLNEALFTRLYTETFHQQYHSYVDFRNAIYLKVAQGLVRMNWLIQYLFGASPRLAVTDTTSRPQRSSVQHPDGRYSQVTGDYTSIDRYVAKLTAAVRQQQLLSVNDFDGPVRLRSNGQLAMMARQGVYYLEYRGLDLDPTSPVGVDANAVAFVRLLASYFVMMPALPAKMVSQVNAQADQLTRQVLGENPTTASAQAVPAVQVLDALADFVKTYGLPNEDAVLLKQLKSWVTDPKKTLSAQIAMQADPLAWALERAARYQESSNERPFELAGFTALDLSSQQLAQQALTRGVQVDVVDPHANILRLTKLGRSQLVVNGSGTDLNPQALTTVLTHKAAAKQILAEHGVPVPASQTYHTANQLIADYDRYVQAGGIVLKAADESHKVIVFRIMPERGLFEQVVRQLFEQTSAVMAEEVVVASSYRFLVIDSRVQAIVERIPANIVGDGRSTVKTLLDRKNGRALRGTAFKWPQSALQLGTIERYRLDSYHLTLDSVVSRGTQILLREDATFGNGADVLDATADMHQSYVQAVEKLVADLHLAVAGVDVMIPNLYAELVPEHPEMAVYLGIHAAPYLYPHLFPMFGTAQPVAGQLLDALFKNED

(19) United States
(12) Patent Application Publication
(10) Pub. No.: US 2008/0194701 A1
(43) Pub. Date: Aug. 14, 2008

(54) Bifunctional Enzyme with Y-Glutamylcysteine Synthetase and Glutathione Synthetase Activity and Uses Thereof

(75) Inventors: Owen W. Griffith, Milwaukee, WI (US); Blythe E. Janowiak, Providence, RI (US)

Correspondence Address: BACON & THOMAS, PLLC, 625 SLATERS LANE, FOURTH FLOOR, ALEXANDRIA, VA, 22314, US

(73) Assignee: Owen W Griffith, Milwaukee, WI (US)

(21) Appl. No.: 11/791,786

(22) PCT Filed: Dec. 8, 2005
(86) PCT No.: PCT/US05/44392

§ 371 Date: Feb. 13, 2008

Related U.S. Application Data
(60) Provisional application No. 60/634,645, filed on Dec. 9, 2004.

(51) Int. Cl. A61K 31/095; C07H 21/04; C12N 9/00; C12N 15/00; C12N 5/00; C12Q 1/00

(52) U.S. Cl. 514/706; 536/23.2; 435/183; 435/320.1; 435/325; 435/455; 435/4

(57) ABSTRACT

Disclosed herein are DNA molecules isolated from Streptococcus agalactiae and other bacterial species encoding a bifunctional enzyme with γ-glutamylcysteine synthetase and glutathione synthetase activities. Also disclosed are bifunctional enzymes with γ-glutamylcysteine synthetase and glutathione synthetase activities, uses of bifunctional enzymes with γ-glutamylcysteine synthetase and glutathione synthetase activities, uses of inhibitors of bifunctional enzymes with γ-glutamylcysteine synthetase and glutathione synthetase activities, and uses of DNA molecules encoding bifunctional enzymes encoding enzymes with γ-glutamylcysteine synthetase and glutathione synthetase activities.

### FIELD OF THE INVENTION

The present invention relates to bifunctional enzymes with γ-glutamylcysteine synthetase and glutathione synthetase activities, to DNA molecules isolated from Streptococcus agalactiae and other bacteria encoding a bifunctional enzyme with γ-glutamylcysteine synthetase and glutathione synthetase activities, to uses of bifunctional enzymes with γ-glutamylcysteine synthetase and glutathione synthetase activities and of the DNAs encoding them, to uses of inhibitors of bifunctional enzymes with γ-glutamylcysteine synthetase and glutathione synthetase activities, and to methods for identifying inhibitors of bifunctional enzymes with γ-glutamylcysteine synthetase and glutathione synthetase activities.

### BACKGROUND OF THE INVENTION

In all previously known prokaryotic and eukaryotic species containing glutathione (GSH, L-γ-glutamyl-L-cysteinylglycine), GSH is synthesized by the sequential action of two separate monofunctional enzymes as follows: (a) γ-glutamylcysteine synthetase catalyzes the ATP-dependent synthesis of L-γ-glutamyl-L-cysteine from L-glutamate and L-cysteine (the enzyme is also known as glutamate-cysteine ligase and is herein referred to as γ-GCS; and the reaction catalyzed is herein referred to as the γ-GCS reaction, represented by Equation 1); and (b) GSH synthetase catalyzes the ATP-dependent synthesis of GSH from L-γ-glutamyl-L-cysteine and glycine (the enzyme is herein referred to as GS; and the reaction catalyzed is herein referred to as the GS reaction, represented by Equation 2). The two enzymes are coded by separate genes, gshA and gshB, respectively, in bacteria and gsh1 and gsh2, respectively, in many eukaryotes. In mammals the γ-GCS reaction is catalyzed by a heterodimeric γ-GCS enzyme comprised of a catalytic (heavy) subunit referred to as glutamate-cysteine ligase catalytic subunit (gene GCLC) and a modifier or regulatory (light) subunit referred to as glutamate-cysteine ligase modifier subunit (gene GCLM).

L-glutamate+L-cysteine+ATP→L-γ-glutamyl-L-cysteine+ADP+Pi (Equation 1)

L-γ-glutamyl-L-cysteine+glycine+ATP→GSH+ADP+Pi (Equation 2)

Hence, by this it has been concluded that both of the enzymes (i.e., γ-GCS and GS), collectively known as GSH synthesis enzymes, are required for the synthesis of GSH from its constituent amino acids.

γ-Glutamylcysteine synthetase and GS have been isolated and characterized from several Gram-negative prokaryotes and from numerous eukaryotes including mammals, amphibians, plants, yeast and protozoa. Glutathione synthesis, catalyzed by the sequential action of γ-GCS and GS, is nearly ubiquitous in eukaryotes where the tripeptide serves both directly and through enzyme-mediated reactions as an antioxidant and as a sacrificial nucleophile useful in the detoxification of reactive electrophiles. Glutathione synthesis is less common among prokaryotes, but distinct γ-GCS and GS enzymes have been isolated and characterized from E. coli and several other Gram-negative species. Although there is substantial evidence that GSH can serve as an antioxidant and sacrificial nucleophile in Gram-negative bacteria, the redundancy of antioxidant defenses and the limited scope of GSH S-transferases in those species suggest GSH may not be required for bacterial survival. For example, E. coli in which γ-GCS has been knocked out exhibit no striking phenotype and show only relatively minor increases in sensitivity to a variety of oxidants.

Glutathione is not known to occur in the archaebacteria, and is rare among Gram-positive bacteria, being identified to date only in some species of Streptococcus, Enterococcus, Lactobacillus and Clostridium. Although some species of Streptococcus (e.g., S. mutans) are thought to take up intact GSH from their medium, it has been reported that Streptococcus agalactiae (S. agalactiae) contains GSH even when grown on GSH-deficient media. Actual synthesis of GSH had not been shown for any Gram-positive bacterium, and the pathway or enzyme(s) involved had not been identified. Furthermore, the gene(s) encoding the enzyme(s) responsible for GSH synthesis had not been isolated and characterized in S. agalactiae or in any other Gram-positive bacteria.

Identification, isolation and characterization of the genes and enzyme(s) responsible for GSH synthesis are important since S. agalactiae and many other Gram-positive bacteria are human pathogens and may depend on GSH for virulence. In particular, S. agalactiae is the leading cause of neonatal meningitis, S. mutans is a major cause of tooth decay and periodontal disease and can cause endocarditis, Enterococcus faecalis (E. faecalis) and Enterococcus faecium (E. faecium) are significant causes of hospital acquired infections, Listeria monocytogenes is a major cause of food poisoning, and Clostridium perfringens is a cause of gangrene. As many antibiotic-resistant strains of these bacteria have developed, it becomes necessary to find new approaches to control the infections they cause. In contrast to the findings in E. coli, there is evidence that GSH has an important role in survival of Gram-positive bacteria. For example, knocking out GSH peroxidase, an enzyme that requires GSH for activity, in Streptococcus pyogenes renders the bacteria less virulent in mice, and it was very recently shown that knocking out the GS activity of the bifunctional GSH synthesis enzyme in Listeria monocytogenes makes those bacteria more susceptible to killing by peroxides and by an activated mouse macrophage cell line.

Accordingly, there is a need for isolation and characterization of the enzymes responsible for GSH synthesis in S. agalactiae and other Gram-positive bacteria, for identification of the genes encoding those enzymes, and for development of pharmacological and other mechanisms for controlling those enzymes or genes for the purpose of treating infections caused by those bacteria.

### SUMMARY OF THE INVENTION

The invention herein is directed to or involves a novel bifunctional enzyme activity that was discovered in S. agalactiae and identified in other mostly Gram-positive bacteria and that was unexpectedly found to catalyze both the γ-GCS and GS reactions and thereby convert L-glutamate, L-cysteine and glycine into GSH in the presence of ATP under suitable reaction conditions. This enzyme is named herein γ-glutamylcysteine synthetase-glutathione synthetase and is abbreviated γ-GCS-GS. The invention also includes isolated DNA molecules corresponding to the genes encoding γ-GCS-GS enzymes (genes denoted herein as gshAB genes), uses of γ-GCS-GS enzymes and gshAB genes, and uses of inhibitors of γ-GCS-GS enzymes, and screening methods for discovering new inhibitors.

In one embodiment, denoted the first embodiment, the present invention discloses an isolated bifunctional enzyme having both γ-GCS and GS activities. In particular examples, the bifunctional enzyme has an amino acid sequence which has a specified degree of identity to any sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26 and SEQ ID NO:28 with or without various N-terminal or C-terminal extensions commonly used to facilitate the purification of proteins (e.g., an N-terminal or C-terminal extension consisting of several histidine residues (His₆-tag, His₈-tag, etc.), GSH S-transferase (GST), and maltose-binding protein (MBP)) and which also still exhibits both γ-GCS and GS activity (i.e., the bifunctional enzyme activity).

In another embodiment, denoted the second embodiment, the present invention discloses a DNA molecule encoding a bifunctional enzyme having both γ-GCS and GS activities. In particular examples, the DNA molecule encoding the bifunctional enzyme has a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27 or a sequence that encodes a protein with an amino acid sequence having a specified degree of sequence identity to any sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26 and SEQ ID NO:28.

In another embodiment, denoted the third embodiment, the present invention discloses a bifunctional enzyme having both γ-GCS and GS activity that is not inhibited by GSH or is only weakly inhibited by GSH. Such enzyme catalyzes the synthesis of GSH from its constituent amino acids without being significantly inhibited by the accumulation of product GSH.

In another embodiment, denoted the fourth embodiment, the present invention discloses expression plasmids containing DNA molecules of the second embodiment that can be used to overexpress γ-GCS-GS in E. coli or other organisms commonly used to overexpress proteins or that can be used to cause expression of γ-GCS-GS in organisms that do not normally contain γ-GCS-GS in order to cause or augment GSH synthesis in those organisms.

In another embodiment, denoted the fifth embodiment, the present invention discloses the use of inhibitors of γ-GCS-GS to limit the synthesis of GSH in microorganisms that contain γ-GCS-GS and that rely on that enzyme for synthesis of their intracellular GSH pool. Such inhibitors have utility as anti-microbial agents and can be used to treat infections in mammals including humans.

In another embodiment, denoted the sixth embodiment, the present invention discloses the use of γ-GCS-GS to synthesize GSH from its constituent amino acids in vitro. In vitro synthesis of GSH using γ-GCS-GS provides a convenient means for the synthesis of GSH. It provides a particularly convenient means for the synthesis of GSH in which one or more of its constituent amino acids is modified structurally or by incorporation of one or more atoms that are relatively uncommon isotopes such as ¹³C, ¹⁴C, ²H, ³H, ¹³N, ¹⁵N, ¹⁷O, ¹⁸O, ³³S or ³⁵S.

In another embodiment, denoted the seventh embodiment, the present invention discloses the use of γ-GCS-GS and of bacteria containing γ-GCS-GS to carry out a high throughput screen for inhibitors of γ-GCS-GS that are effective in vitro and in vivo.

These embodiments, together with other aspects of the present invention, and with the various features of novelty that characterize the invention, are pointed out with particularity in the claims annexed hereto that form a part of this disclosure. For a better understanding of the invention, its operating advantages, and the specific objects attained by its uses, refer to the accompanying drawings and descriptive matter that illustrate exemplary embodiments/examples of the invention.

### BRIEF DESCRIPTION OF THE DRAWINGS

The advantages and features of the present invention will become better understood with reference to the following, more detailed description and claims, taken in conjunction with the accompanying drawings, in which:

FIG. 1A is a schematic of the bifunctional enzyme (i.e., γ-GCS-GS) isolated from S. agalactiae, showing the N-terminal region that is homologous to E. coli γ-GCS and the C-terminal remainder of the protein that is homologous to E. coli D-Ala, D-Ala ligase and discovered to account for GS activity;

FIG. 1B illustrates a phylogenetic tree based on amino acid sequences of γ-GCS-GS enzymes showing the relatedness of the sequences in the bacteria shown;

FIG. 1C is a schematic of the bifunctional enzyme (i.e., γ-GCS-GS) showing that the N-terminal region that is homologous to E. coli γ-GCS and the C-terminal region that is homologous to E. coli D-Ala, D-Ala ligase and discovered to have GS activity actually overlap by about 160 amino acids.

FIG. 1D illustrates an alignment of amino acid sequences for γ-GCS-GS enzymes from 14 species. The 57 amino acid residues that are conserved in all of the sequences are shown in bold. The species shown are Mannheimia succiniciprodecens (Ms), Pasteurella multocida (Pm), Haemophilus somnus (Hs), E. faecium (Efm), E. faecalis (Efs), S. mutans (Sm), Streptococcus suis (Ss), S. agalactiae (Sa), Steptococcus thermophilus (St), Desulfotalea psychrophila (Dp), Clostridium perfringens (Cp), Listeria monocytogenes (Lm), Listeria innocua (Li), and Lactobacillus plantarum (Lp).

FIG. 2 illustrates a phylogenetic tree showing that there are four distinct superfamilies of enzymes having γ-GCS activity;

FIG. 3 illustrates a phylogenetic tree showing that there are two distinct superfamilies of enzymes having GS activity;

FIG. 4 is a graph showing the amount of γ-glutamyl-α-amino[¹⁴C]butyrate synthesized per mg of protein added to the reaction mixture plotted as a function of time;

FIG. 5 is a photo of a SDS-PAGE gel of purification fractions for endogenous S. agalactiae γ-GCS-GS;

FIG. 6 is a photo of a SDS-PAGE gel of purification fractions for S. agalactiae His₈-γ-GCS-GS expressed in SG13009 [pRARE] E. coli; and

FIG. 7 is graph of the formation of GSH by S. agalactiae γ-GCS-GS from its constituent amino acids as a function of time.

### DETAILED DESCRIPTION OF THE INVENTION

The exemplary embodiments/examples described herein provide detail for illustrative purposes, and it is understood that various omissions, or substitutions of equivalents are contemplated as circumstances may suggest or render expedient.

The present invention illustrates that crude extracts of Streptococcus agalactiae (S. agalactiae) catalyze the γ-GCS and GS reactions (represented by Equations 1 and 2) and can synthesize GSH from its constituent amino acids. Both intact S. agalactiae and homogenates of those cells are clearly able to synthesize GSH as determined by a highly specific enzymatic recycling assay for total GSH and by the glutamate- and cysteine-dependent incorporation of radiolabeled [¹⁴C]glycine into an anionic peptide that binds to an ion-exchange resin (e.g., Dowex 1) and elutes under standard conditions used to elute GSH from such resins. Furthermore, it was possible to purify γ-GCS activity from S. agalactiae homogenates, showing that GSH synthesis in S. agalactiae proceeds through the initial synthesis of γ-glutamylcysteine. Preparations of the γ-GCS activity from S. agalactiae were subjected to SDS-PAGE, in-gel trypsin digestion and Matrix-Assisted Laser Desorption Ionization-Time of Flight (MALDI-TOF) sequencing to provide sufficient amino acid sequence information to directly identify the gene in S. agalactiae that encodes the protein having γ-GCS activity.

The γ-GCS-GS gene (herein referred to as SAG1821 or gshAB) was identified and cloned, and the corresponding protein (i.e., enzyme) was expressed and purified. It was found that the isolated enzyme catalyzes both the γ-GCS and GS reactions (represented by Equations 1 and 2), thereby behaving as a bifunctional enzyme for GSH synthesis. Enzyme purified from E. coli engineered to overexpress the bifunctional γ-GCS-GS enzyme of S. agalactiae exhibited γ-GCS activity having a specific activity under optimal reaction conditions of about 1300 units per mg of protein and GS activity having a specific activity of about 2000 units per mg of protein, here 1 unit is the amount of enzyme activity required to catalyze the synthesis of 1 μmol of product in 1 hour.

Thus, the present invention provides an isolated, novel bifunctional enzyme having γ-GCS and GS activities, and an isolated gene (i.e., the DNA molecule) that encodes the bifunctional enzyme. The bifunctional enzyme consists of an amino acid sequence which has a specified degree of identity to any of the sequences designated by SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, and SEQ ID NO:28 and which has both γ-GCS and GS activity. The isolated DNA molecule encoding the bifunctional enzyme consists of a nucleotide sequence having a specified degree of identity to any of the sequences designated by SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27. The enumerated amino acid sequences and nucleotide sequences are listed in the Sequence Listing Section.

As used herein, the bifunctional enzyme is referred to as γ-GCS-GS. The γ-GCS-GS enzymes encoded by or present in S. agalactiae, S. mutans, and E. faecalis (each enzyme being a γ-GCS-GS isoform) have been characterized in terms of catalytic activity, substrate specificity, and inhibition by GSH, transition-state analog sulfoximines, and other inhibitors, and those results have been compared to similar determinations for known monofunctional γ-GCS and GS enzymes.

Disregarding any N— or C-terminal extensions added to facilitate protein purification, all γ-GCS-GS isoforms have a total molecular mass of about 85 kDa (range 80 to 92 kDa) and are comprised of about 750 amino acids (range 725 to 800 amino acids). The N-terminal approximately 520 amino acids of γ-GCS-GS (herein referred to as the γ-GCS domain) show significant homology with known monofunctional γ-GCS protein sequences, and, in particular, the S. agalactiae γ-GCS-GS isoform shows 32% identity and 43% similarity with E. coli γ-GCS (relative molecular mass (Mᵣ) about 56,000, 520 amino acids), whereas the C-terminal approximately 230 amino acids of γ-GCS-GS show no significant homology with any known monofunctional GS protein sequence as determined using the BLAST algorithm to search all bacterial genomes. Furthermore, the C-terminal approximately 390 amino acid sequence (residues from about 360 through about 750, herein referred to as the ATP-grasp protein domain or the GS domain) is homologous to E. coli D-Ala, D-Ala ligase, having in the case of S. agalactiae γ-GCS-GS 23% identity and 37% similarity to that enzyme. D-Ala, D-Ala ligase does not have GS activity, but it does have a protein fold similar to known GS proteins. Since that protein fold, called an ATP-grasp domain, is shared by at least 17 proteins, it was not possible to predict from the sequence what activity, if any, might be catalyzed by the C-terminal domain of γ-GCS-GS. Since that ATP-grasp domain sequence overlapped with the N-terminal sequence having similarity to E. coli γ-GCS and that overlap resulted in numerous amino acids in the overlap region being different from those present in E. coli γ-GCS, it was not possible to predict with certainty that the N-terminal domain of γ-GCS-GS catalyzed the γ-GCS reaction.

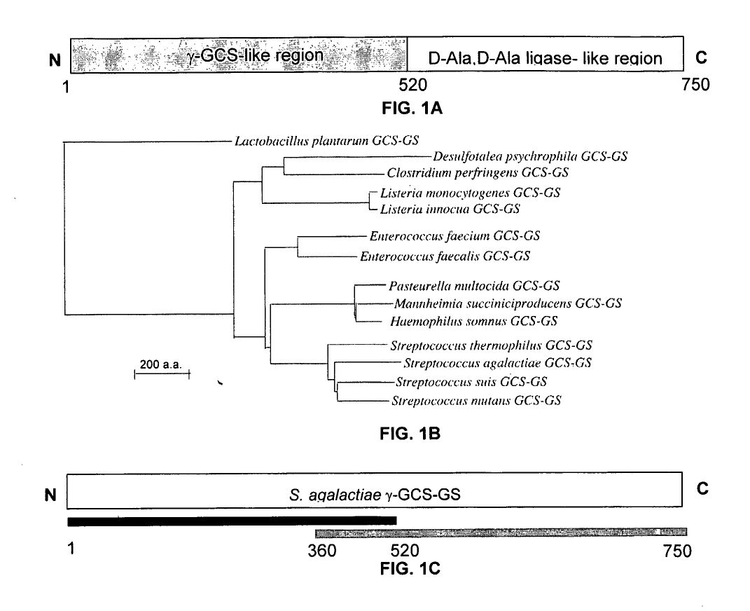

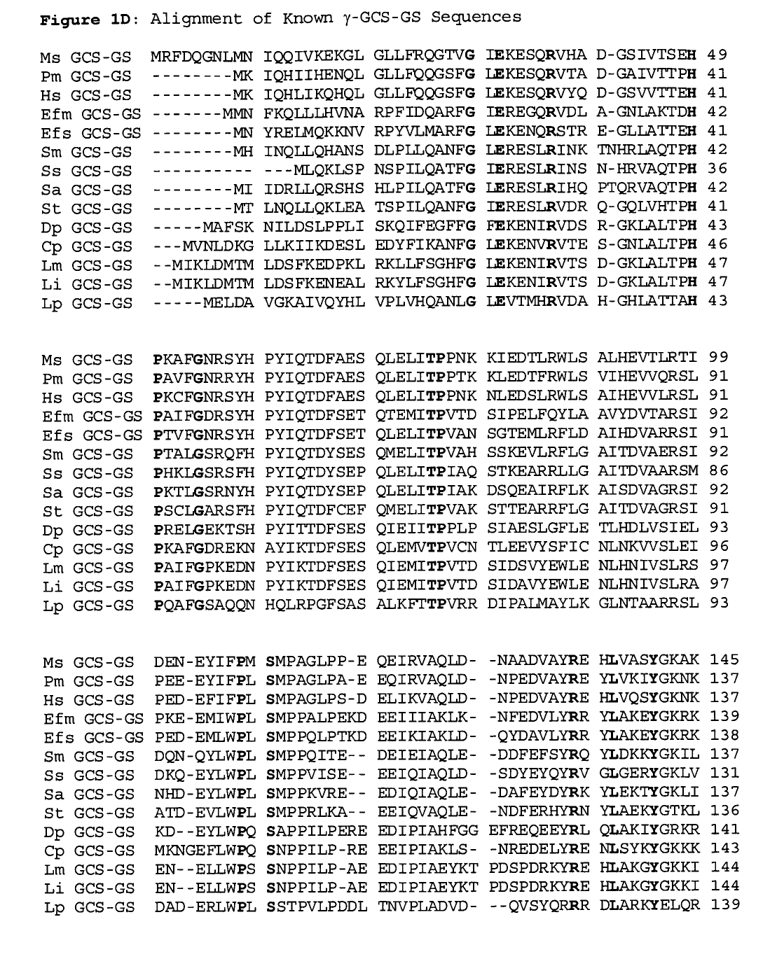

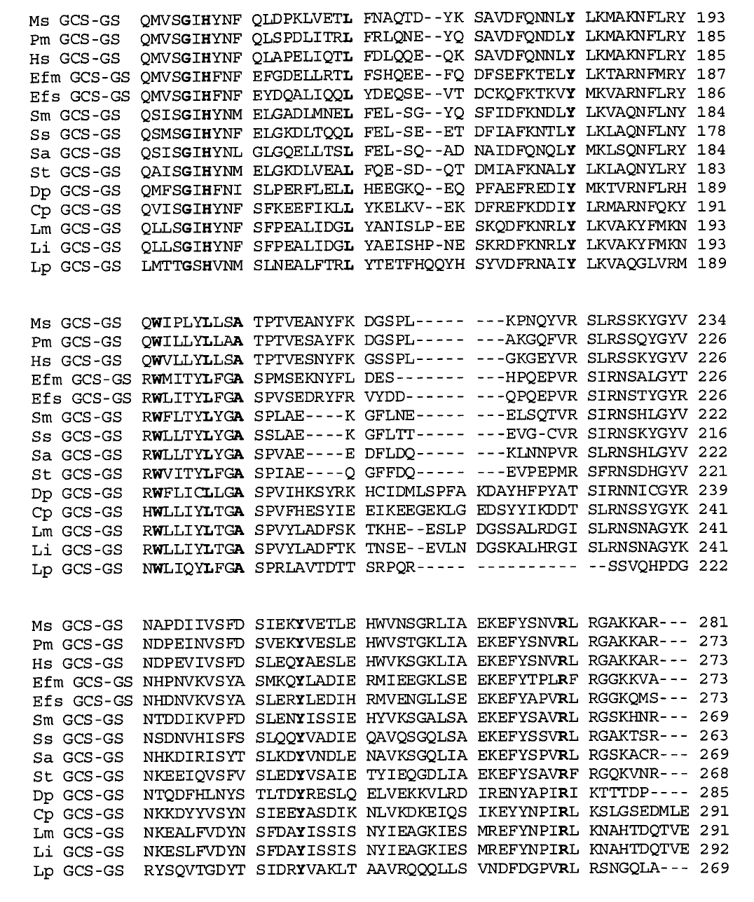

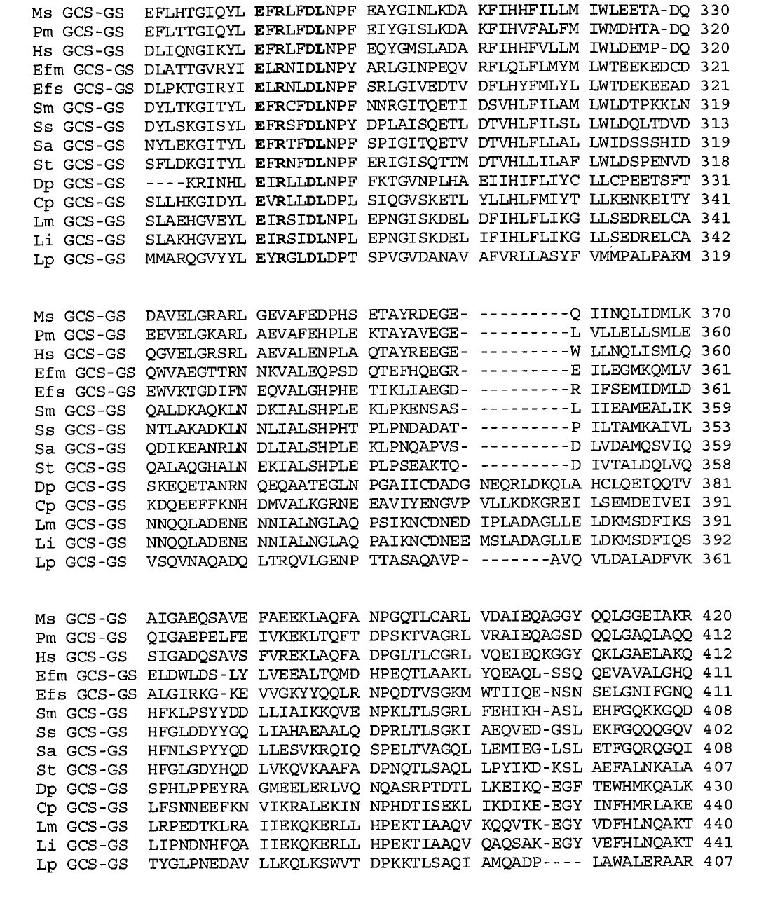

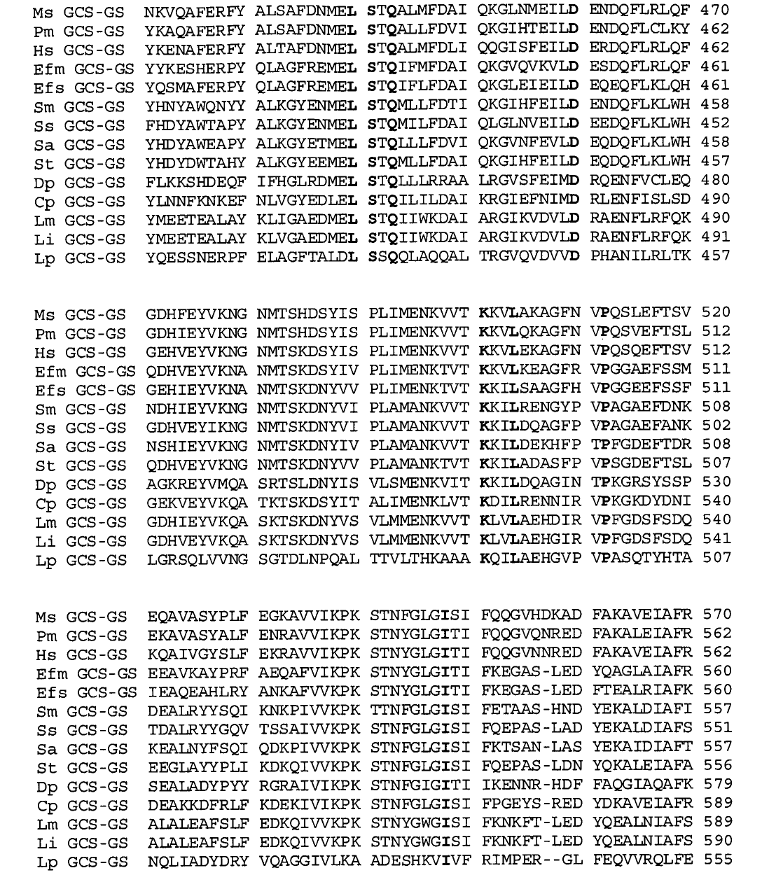

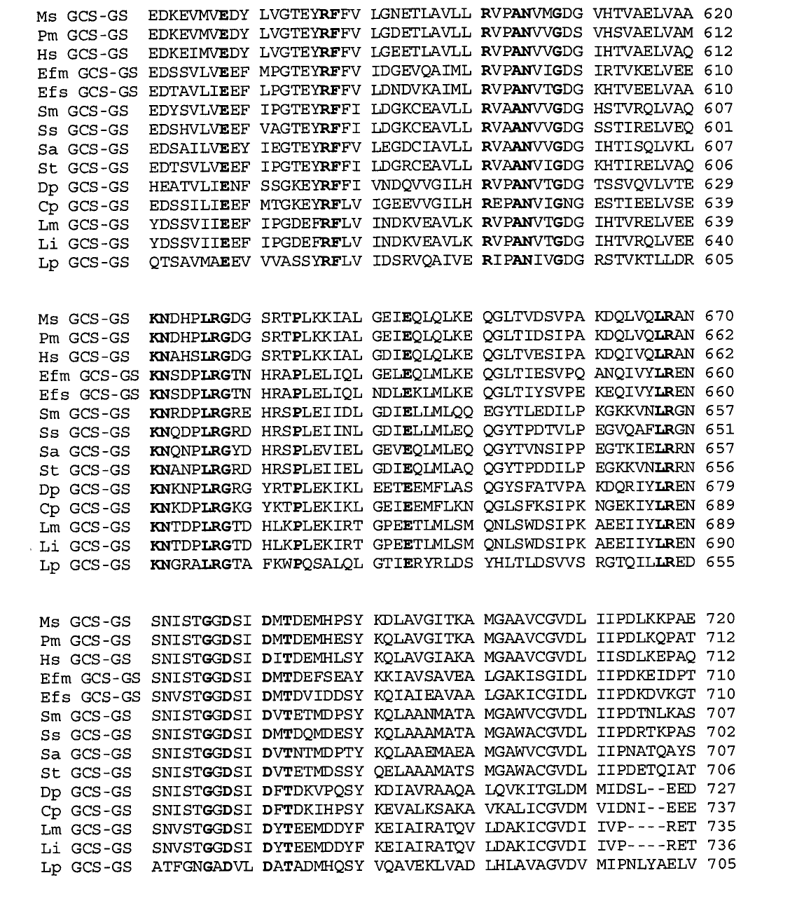

A schematic of the bifunctional enzyme (i.e., γ-GCS-GS) is shown in FIG. 1A, in which the portions originally attributed to the γ-GCS domain and the ATP-grasp protein domain, later identified as part of the GS domain, are based on homology with E. coli γ-GCS and D-Ala, D-Ala ligase, respectively. The homologous regions are represented as ‘γ-GCS-like region’ (N-terminal or amino terminal domain) and as ‘D-Ala, D-Ala ligase-like region’ (C-terminal or carboxyl terminal domain), respectively. Based on those homologies, the domains actually overlap by about 160 amino acids (FIG. 1C). Key structural and functional features of the γ-GCS-GS bifunctional enzyme are these: (i) an amino acid sequence comprised of about 750 amino acids and a relative molecular mass (Mᵣ) of about 85,000, (ii) an N-terminal sequence of approximately 500 amino acids that is homologous to E. coli γ-GCS, (iii) a C-terminal sequence of approximately 350 amino acids that forms an ATP-grasp domain, and (iv) ability to catalyze both the γ-GCS reaction and the GS reaction. Other features of amino acid sequence similarity among the family of γ-GCS-GS proteins are illustrated in FIG. 1D, which shows an alignment of γ-GCS-GS sequences from 14 bacterial species. Fifty seven amino acid residues that align in all 14 sequences and are thus conserved in all sequences are shown in bold. Identity of at least 50 of these 57 residues (88%) in new γ-GCS-GS sequences is indicative that the sequence is a member of the γ-GCS-GS protein family. In addition to the fully conserved residues, there are many additional residues conserved in 13 of the 14 sequences listed. Identity of a majority of those residues in new γ-GCS-GS sequences is indicative that the sequence is a member of the γ-GCS-GS protein family.

For the purpose of facilitating purification it is common practice to engineer expression plasmids to encode the native protein plus an N-terminal or C-terminal extension that binds reversibly to a solid support. Such extensions include but are not limited to poly-histidine sequences (e.g., His₆ or His₈), which bind to resins displaying a bound Ni²⁺ ion, GSH S-transferase (GST), which binds to resins displaying GSH, and maltose binding protein (MBP), which binds to resins displaying maltose polymers such as amylose. Incorporation of any N-terminal or C-terminal extensions intended to facilitate purification increases the molecular mass and amino acid number of γ-GCS-GS by an amount equal to the mass and amino acid number of the extension(s). Such extensions are disregarded in evaluating whether putative γ-GCS-GS amino acid or gene sequences meet the criteria for being members of the γ-GCS-GS enzyme family.

Without being bound by theory, it is believed that the N-terminal domain of the γ-GCS-GS protein accounts for the observed γ-GCS activity of the bifunctional enzyme and that the C-terminal domain of the γ-GCS-GS protein accounts for the observed GS activity.

To investigate the possible presence of the γ-GCS-GS-dependent GSH synthesis pathway in other organisms, the S. agalactiae gene sequence originally designated SAG1821 was blasted against the NCBI bacterial genome databases. As shown in FIG. 1B, highly homologous sequences were identified in 13 species, in addition to S. agalactiae: S. mutans, Streptococcus suis, Steptococcus thermophilus, E. faecalis, E. faecium, Listeria innocua, Listeria monocytogenes, Clostridium perfringens, Desulfotalea psychrophila, Pasteurella multocida, Mannheimia succiniciprodecens, Haemophilus somnus, and Lactobacillus plantarum. Pasteurella multocida, Mannheimia succiniciprodecens, and Haemophilus somnus are Gram-negative bacteria; all of the others are Gram-positive bacteria. All of the bacteria listed are potential human pathogens except Desulfotalea psychrophila. As additional bacterial genomes become available, additional sequences homologous to those shown in FIG. 1B can easily be identified using the same BLAST search technique. Taken together, these results indicate that γ-GCS-GS has a broad, albeit sparse, distribution among bacteria that are mostly human pathogens and is coded by a novel gene that is designated as gshAB in analogy to the designation of the monofunctional γ-GCS and GS bacterial genes as gshA and gshB, respectively.

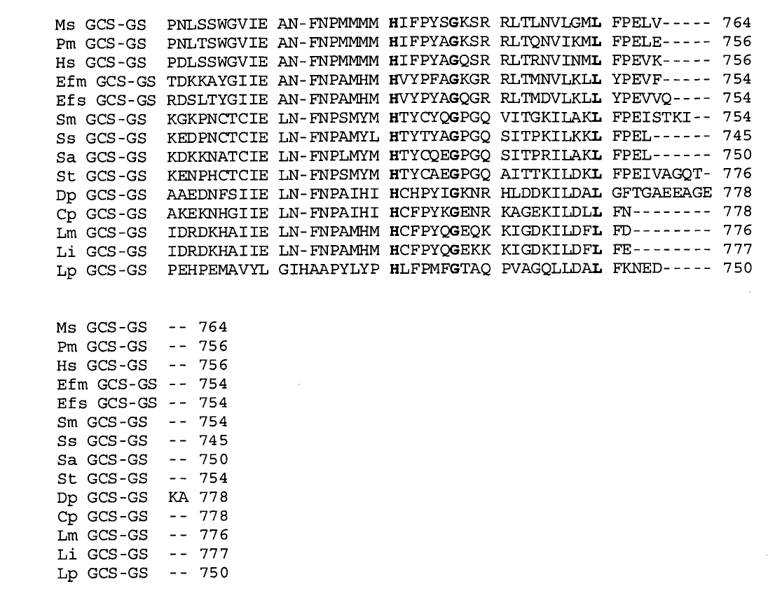

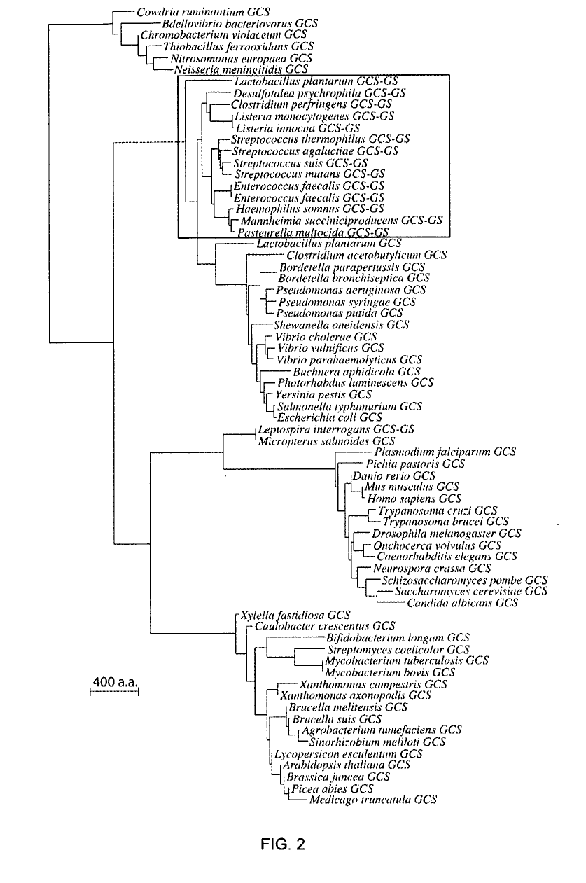

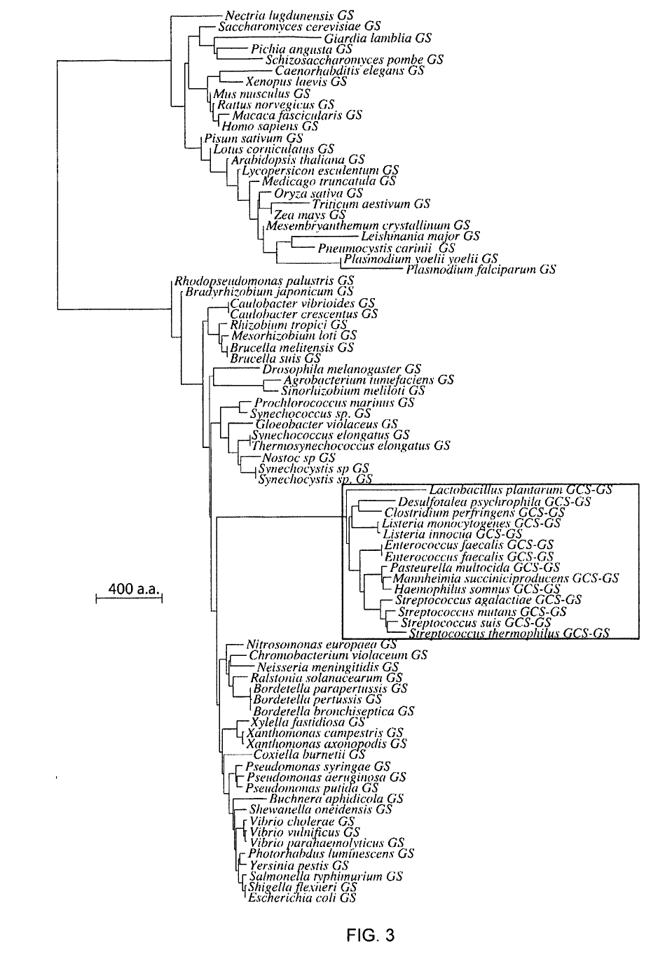

Based on their amino acid sequences, the 14 highly homologous bifunctional γ-GCS-GS isoforms (i.e., the γ-GCS-GS from the 14 species shown in FIG. 1B) were aligned phylogenetically with known monofunctional γ-GCS enzymes (FIG. 2) and GS enzymes (FIG. 3). The alignments were made based on the putative γ-GCS domain of γ-GCS-GS (residues 1 to about 520) (FIG. 2) and the putative GS domain of γ-GCS-GS (residues about 360 to about 750) (FIG. 3). About 160 amino acids (residue ˜360 to ˜520) had to be attributed to both domains for purposes of these alignments, indicating that the domains overlap as shown in FIG. 1C and indicating that the activity of neither domain could be reliably predicted from the genome sequence information alone. As shown in FIG. 2 and FIG. 3, the γ-GCS-GS amino acid sequences from S. agalactiae and the other 13 highly homologous enzymes from other bacteria partially fit into both groups of enzymes, but clearly represent their own family (boxed in the figures). S. agalactiae is highlighted in FIG. 2. It has previously been found that prokaryotic and eukaryotic γ-GCS enzymes, along with the mechanistically-related glutamine synthetases, represent a γ-GCS and glutamine synthetase superfamily comprised of four γ-GCS families and three glutamine synthetase families. By this analysis, the putative γ-GCS-GS enzymes (i.e., γ-GCS-GS from S. agalactiae and the other 13 highly homologous enzymes from other bacteria) group into the Prokaryote III γ-GCS family but are distinct from known members of that family, as shown in FIG. 2. In contrast to the γ-GCS enzymes, prokaryotic and eukaryotic GS sequences are so divergent that it is presently uncertain whether they are homologous or are products of convergent evolution. As previously described, the GS domain of γ-GCS-GS is homologous to known D-Ala, D-Ala ligase sequences but is only very weakly related to any known GS. Both D-Ala, D-Ala ligase and GS are ATP-grasp proteins, and the C-terminal sequence of γ-GCS-GS is an ATP-grasp domain. As shown in FIG. 3, the GS domain of the γ-GCS-GS family can be grouped with the prokaryotic GS sequences but only as a distinct branch that diverges very early (the γ-GCS-GS family is boxed in FIG. 3 and S. agalactiae is highlighted). Without being bound by theory, it is believed that the GS domain of γ-GCS-GS was, in fact, acquired by gene duplication of D-Ala. D-Ala ligase and that that domain evolved to have GS activity after γ-GCS-GS separated from the other Prokaryotic III family γ-GCS enzymes, as shown in FIG. 2.

We expect that other γ-GCS-GS proteins will be found. New γ-GCS-GS proteins can be characterized by their bifunctional enzymatic activity (i.e., γ-GCS and GS activities), their size range (Mᵣ of about 80,000 to 92,000), the inclusion of an amino terminal region that has significant sequence identity (greater than 30%) to a native monofunctional γ-GCS enzyme, and a C-terminal region with an ATP grasp domain. It is expected from the data presented here that the other γ-GCS-GS enzymes will have at least a 27% sequence identity with SEQ ID NO:2, and will likely have at least a 34% sequence identity with SEQ ID NO:2. If the novel γ-GCS-GS is from a related Streptococcus species, the sequence is expected to be at least 60% identical. In addition to sequence identity, other γ-GCS-GS will maintain the dual enzymatic activity (i.e., have both γ-GCS and GS activity).

With respect to the interactions of γ-GCS-GS with substrates, the γ-GCS and GS activities of S. agalactiae γ-GCS-GS show both similarities to and differences from previously reported γ-GCS and GS enzymes. The γ-GCS activity of S. agalactiae γ-GCS-GS is similar to E. coli γ-GCS with respect to its Kₘ values for L-cysteine and ATP. In contrast, γ-GCS-GS has a markedly lower affinity for L-glutamate and L-α-aminobutyrate, a L-cysteine analog that commonly can replace L-cysteine as a substrate in γ-GCS reactions (see Example 5, Table 3). Since L-α-aminobutyrate is not a physiological substrate, low affinity for that L-cysteine surrogate was not particularly surprising, and there is no obvious evolutionary pressure to preserve a cysteine active site with high affinity for L-α-aminobutyrate. Low affinity for L-glutamate, on the other hand, was initially surprising, because glutamate is clearly the physiological substrate, based on the relative inactivity of glutamate analogs and the observation that S. agalactiae contain genuine GSH, as established by both a highly specific enzymatic recycling assay (see Example 1) and earlier studies using high resolution HPLC to detect biological thiols. Many Gram-positive bacteria, including S. agalactiae, have been reported to maintain exceptionally high intracellular concentrations of L-glutamate (60-100 mM), and it is likely that those concentrations allow GSH synthesis to proceed efficiently despite the high Kₘ for L-glutamate.

The amino acid sequence of the GS domain of S. agalactiae γ-GCS-GS is related to D-Ala, D-Ala ligase rather than GS, but known monofunctional GS enzymes and D-Ala, D-Ala ligase enzymes all belong to the ATP-grasp superfamily and therefore have similar folds. Absence of significant sequence homology between the GS domain of γ-GCS-GS and known GS enzymes meant that there was no expectation that Vₘₐₓ and substrate Kₘ values would be similar, and, in fact, S. agalactiae GS activity exhibits a specific activity that is about three-fold to about six-fold higher than reported for any known GS enzyme (See Example 5, Table 4). Taking into account the apparently small size of the GS domain of γ-GCS-GS (estimated 31-40 kDa) relative to the sizes of known monofunctional GS enzymes (E. coli, 36 kDa; human, 52 kDa; yeast, 56 kDa), the catalytic efficiency of the S. agalactiae GS domain is about 5.3-fold to about 18-fold greater than that seen with known monofunctional GS enzymes.

With respect to substrate binding, the Kₘ value of the S. agalactiae GS domain for ATP is within the range of values previously reported for known GS enzymes, but the Kₘ values for glycine, L-γ-glutamyl-L-α-aminobutyrate and L-γ-glutamyl-L-cysteine are significantly higher than other known GS enzymes. Since L-γ-glutamyl-L-α-aminobutyrate is not a physiological substrate, its high Kₘ is not intrinsically surprising. The relatively high Kₘ for L-γ-glutamyl-L-cysteine is less easily rationalized.

With respect to their potential inhibition by GSH, the bifunctional S. agalactiae γ-GCS-GS enzyme is different from all known monofunctional γ-GCS and GS enzymes. In all previously described examples, GSH synthesis was found to be regulated in part by feedback inhibition of γ-GCS by GSH (GSH acted as a non-allosteric feedback, product inhibitor). In contrast, it was found that GSH does not significantly inhibit either the γ-GCS activity or the GS activity of S. agalactiae γ-GCS-GS when using GSH concentrations of up to 100 mM, whereas human γ-GCS and E. coli γ-GCS were potently inhibited even at lower levels of GSH (see Example 7, Table 5). Accordingly, S. agalactiae maintain a much higher intracellular GSH concentration than E. coli, despite the fact that γ-GCS activity is lower in S. agalactiae homogenates. For example, the GSH concentration in S. agalactiae was 304±11 nmol per mg protein whereas the GSH concentration in E. coli was 19±3 nmol per mg protein (see Example 1). The high levels of GSH may be rationalized by the fact that S. agalactiae lack the antioxidant enzyme catalase and it is thus advantageous for S. agalactiae to accumulate GSH, an alternative, non-enzyme antioxidant. It was observed that the high Kₘ for L-glutamate may provide an alternative regulation of GSH synthesis under conditions of limiting nutrients, wherein the glutamate concentration decreases to a level that limits γ-GCS activity, preventing depletion of, for example, free L-cysteine. The fact that S. agalactiae γ-GCS-GS is not inhibited by GSH makes it particularly useful both in vivo and in vitro as a catalyst for GSH synthesis since GSH synthesis can proceed to form high concentrations without feedback inhibition by GSH. The fact that S. agalactiae γ-GCS-GS has a high Kₘ for L-glutamate makes it particularly useful in vivo as a catalyst for GSH synthesis because regulation of GSH synthesis occurs through a mechanism other than accumulation of GSH (i.e., presence of S. agalactiae γ-GCS-GS in cells, either naturally or through genetic engineering using the DNA encoding S. agalactiae γ-GCS-GS, results in the desirable accumulation of GSH to high levels but the high Kₘ for L-glutamate assures that synthesis will still be regulated by availability of L-glutamate and the intracellular pool of that amino acid will not be depleted to levels that compromise other L-glutamate-dependent reactions and thereby prevent proper functioning of the cell).

The bifunctional γ-GCS-GS enzymes of E. faecalis and S. mutans were found to differ from the γ-GCS-GS of S. agalactiae in that they are inhibited by GSH. Correspondingly, E. faecalis was found to maintain a lower intracellular GSH level that S. agalactiae. The fact that the γ-GCS-GS of E. faecalis and S. mutans are inhibited by GSH makes them useful as in vivo catalysts for the synthesis of GSH in cells where it is desirable to have GSH autoregulate its own synthesis. For example, the DNA encoding the γ-GCS-GS of E. faecalis (i.e., E. faecalis gshAB) can be inserted into cells that do not normally contain enzymes for synthesizing GSH to create cells that maintain a moderate level of GSH. Because both of the enzyme activities required for GSH synthesis are present on a single gene, use of gshAB is advantageous over the combined use of previously known gshA and gshB genes, which would require that both genes be inserted into and comparably expressed in an organism where GSH synthesis was desired.

With respect to their inhibition by transition state analogs including certain sulfoximines (e.g., buthionine sulfoximine (BSO)) the bifunctional γ-GCS-GS enzymes exhibit both similarities to and differences from previously described monofunctional enzymes involved in GSH synthesis. Thus, S-alkyl-L-homocysteine sulfoximines are well known inhibitors of monofunctional γ-GCS enzymes. Inhibition of γ-GCS is typically better with buthionine sulfoximine (BSO, S-butyl-L-homocysteine sulfoximine, represented by Structure I) than with S-alkyl-L-homocysteine sulfoximines having smaller S-alkyl groups. The γ-GCS activity of γ-GCS-GS is inhibited by L-buthionine-S-sulfoximine (L-S—BSO, the active diastereomer of BSO), albeit less effectively than E. coli or mammalian γ-GCS. Other known γ-GCS inhibitors include methionine sulfoximine (MSO, Structure II), 2-amino-4-phosphonobuytric acid (APB, Structure III), 2-amino-5-phosphonovaleric acid (APV, Structure IV), glufosinate ammonium (structure V), and 1-aminocyclopentane-1,3-dicarboxylic acid (ACPD, Structure VI). These inhibitors are all analogs of one or more of the γ-GCS-GS substrates. MSO inhibits both glutamine synthetase and γ-GCS, whereas BSO is γ-GCS selective. Although both E. coli and mammalian γ-GCS are inhibited by MSO, it was found that L-SR-MSO causes no significant inhibition of the γ-GCS activity of S. agalactiae γ-GCS-GS even when pre-incubated with the enzyme in the presence of MgATP and the absence of L-glutamate, conditions that favor binding and phosphorylation of the inhibitor. Of the other inhibitors listed, glufosinate and APB were found to be moderately good inhibitors, whereas APV was not effective.

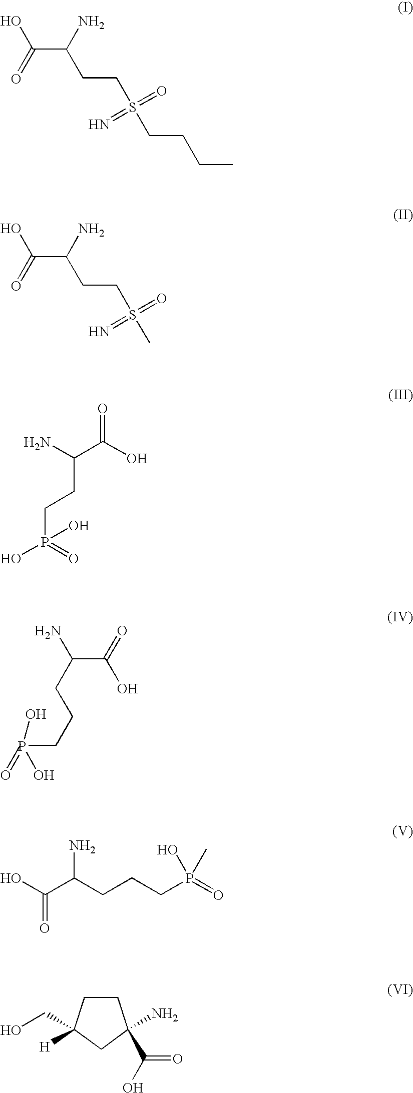

With respect to the use of γ-GCS-GS inhibitors to treat infections in mammals including humans, the γ-GCS-GS inhibitor is administered in a dose sufficient to decrease the rate of GSH synthesis in the infecting bacteria and thereby cause the bacteria to maintain a lower intracellular concentration of GSH. Such bacteria are less virulent and are more easily and quickly cleared by the immune system of the treated animal. Infections that include any or several of S. agalactiae, S. mutans, Streptococcus suis, Steptococcus thermophilus, E. faecalis, E. faecium, Listeria innocua, Listeria monocytogenes, Clostridium perfringens, Pasteurella multocida, Mannheimia succiniciprodecens, Haemophilus somnus, or Lactobacillus plantarum are treatable with γ-GCS-GS inhibitors. Inhibitors of γ-GCS-GS may be administered orally or parenterally (e.g., by intravenous, intramuscular, intraperitoneal or subcutaneous injection), or may be applied topically. The oral route is preferred for systemic infections. In general the dose of γ-GCS-GS inhibitor ranges from 1 μg to 10 g per kg, often 10 μg to 1 g per kg, most often 100 μg to 100 mg per kg of the mammal's body weight per day. Administration of γ-GCS-GS inhibitor is continued until signs of infection are absent and is preferably continued for an additional 3 to 6 days to assure there is no recurrence and to avoid development of resistant strains of bacteria. Optionally, an agent that induces oxidative stress in the infecting bacteria may be coadministered with the inhibitor of γ-GCS-GS. Because γ-GCS-GS inhibitors decrease the concentration of GSH in infecting bacteria and because infecting bacteria rely on GSH as a defense against oxidative stress, coadministration of an agent that increases oxidative stress in the infecting bacteria increases the antibacterial cytotoxic effect of GSH depletion. Agents that increase oxidative stress in the infecting bacteria include various redox cycling drugs and drugs that interfere with electron transport in bacteria (e.g., nitrofurantoin, ampicillin plus gentamicin, 2-hydroxy-N-(3,4-dimethyl-5-isoxazolyl)-1,4-naphthoquinone-4-imine, and many quinines and hydroquinones).

With respect to the high throughput screening of compounds to identify γ-GCS-GS inhibitors, sets of compounds (e.g., combinatorial libraries) are first screened as inhibitors of isolated γ-GCS-GS using enzyme isolated from the bacterial species of interest. The screen is carried out using multi-well plates in which each well contains a reaction mixture suitable for GSH synthesis by the γ-GCS-GS isoform of interest, 1 ng to 10 mg samples of the compound(s) to be tested as inhibitor, and γ-GCS-GS, which is added last to start the reaction. After a fixed period of time, a small portion of the solution in each well is transferred to the corresponding well of a second plate in which is present a solution suitable for detection of GSH. Preferably the solution in the wells of the second plate is a reaction mixture similar to that described in O. W. Griffith Anal. Biochem. 106, 207-212 (1980) and that allows the quantitation of GSH using a GSSG reductase-dependent GSSG to GSH recycling assay. Compounds that show significant inhibition of isolated γ-GCS-GS, preferably >50% inhibition at a concentration <100 μg/ml are screened for their ability to inhibit GSH synthesis in intact bacteria. That screen is also carried out in multi-well plates in which each well contains a small and approximately equal number of bacteria in a suitable growth medium, such medium preferably having no GSH or GSSG, and 1 ng to 10 mg samples of one or several of the individual compounds to be tested as inhibitors. The bacteria are then allowed to grow in the wells and after a suitable period, preferably 6 to 48 hrs, the bacteria are sedimented in the wells by centrifugation, and the supernatant medium is removed. The bacteria are then resuspended and broken, preferably by resuspension in a solution containing lysozyme, and GSH in the resulting solution is determined by adding to the wells a reaction mixture suitable for the determination of GSH. Preferably, the solution for determination of GSH is a solution of a GSSG reductase-containing reaction mixture similar to that used for determination of GSH formed in the screening procedure for inhibitors of isolated γ-GCS-GS.

The present invention is further illustrated by the following non-limiting examples:

### EXAMPLES

For the following examples, the biochemical reagents were obtained from Sigma unless indicated otherwise. The bacterial strains were obtained as follows: an expression strain of E. coli, SG13009, from Qiagen; a sequenced strain of S. agalactiae, 2603 V/R S. agalactiae, from ATCC (ATCC #BAA-611); E. faecalis 10C1, from ATCC (ATCC #19434); Streptococcus mutans NIDR 6715-15, from ATCC (ATCC #25175); E. faecium NCTC 7171, from ATCC (ATCC #11700). E. coli plasmids were obtained as follows: pCR2.1 from Invitrogen, pREP4 and pQE30 from Qiagen, pRARE from Promega, pQE30T from F. C. Peterson (Peterson, F. C., et al, J. Biol. Chem. 279, 12598-12604 (2004)). Detailed experimental procedures are provided in B. E. Janowiak and O. W. Griffith (J. Biol. Chem. 280, 11829-11839 (2005)), the whole of which, along with the provisional application for this case (60/634,645), is incorporated by reference.

### Example 1

This example demonstrated that S. agalactiae, E. faecalis, and E. faecium synthesize GSH and that S. agalactiae have γ-GCS activity.

Fifty ml cultures were grown with gentle agitation (orbital shaker) in appropriate media (Todd-Hewitt broth supplemented with 2% yeast extract (THY) for S. agalactiae and yeast-tryptone medium (2×YT) for E. coli used as controls) for about 18 hours, and cells were harvested by centrifugation and washed twice by suspension in 1 ml of phosphate-buffered saline (PBS) followed by re-centrifugation. The cell pellet was then re-suspended in 500 μl of 20 mg/ml lysozyme in PBS, and cells were broken by sonication (3 pulses of 30 seconds each) on ice. Crude homogenates were clarified by centrifugation, and 20 μl of 50 percent 5′-sulfosalicyclic acid was added to a 200 μl aliquot of the supernatant to precipitate protein. Precipitated proteins were removed by centrifugation, and the supernatant was assayed for total GSH using a modified version of the ‘Tietze assay,’ a well-established GSSG reductase-dependent enzymatic recycling assay for total GSH (total GSH is defined as GSH+2× GSSG, where GSSG is the disulfide of GSH) (O. W. Griffith Anal. Biochem. 106, 207-212 (1980)). Using three independent cultures, the total GSH concentration in S. agalactiae was found to be 304±11 nmol per mg of protein. For comparison, the total GSH concentration in E. coli was 19±3 nmol per mg of protein. Also, it was found that total GSH levels in S. agalactiae were about three-fold lower in unagitated (i.e., less aerobic) cultures.

In order to verify that S. agalactiae could synthesize GSH, rather than simply take up GSH or GSSG that were present in the growth media, extracts of the bacteria were assayed for GSH levels after the bacteria were grown for about 18 hours in chemically-defined medium that lacked GSH and GSSG. The composition of the media was as described by N. P. Willett and G. E. Morse (J. Bacteriol. 91, 2245-2250 (1966)). Other than changing the growth medium (i.e., to a chemically-defined medium lacking GSH and GSSG), the studies were identical to those described above for determining total GSH levels. The total GSH concentration was 327±12 nmol per mg of protein for three independent, agitated cultures. Since there was no GSH or GSSG in the growth medium, this study shows S. agalactiae were able to synthesize GSH.

In a similar study, S. agalactiae, E. faecalis and E. faecium were separately grown for 24 hours in chemically defined medium lacking GSH and GSSG. Total GSH levels (nmol per mg protein) in aerobic (i.e., agitated) cultures were as follows: S. agalactiae, 469±11; E. faecalis, 78±4; E. faecium, 189±10. This example confirmed that S. agalactiae can synthesize GSH and showed two pathologically important enterococcal species that encode γ-GCS-GS in their genomes can also synthesize GSH.

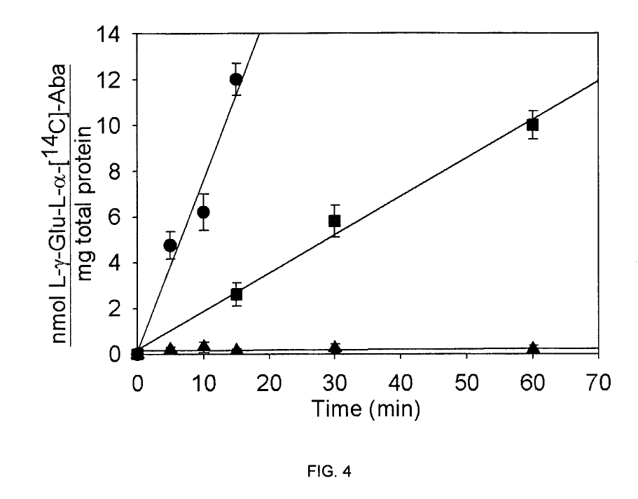

To determine if S. agalactiae could carry out the γ-GCS reaction, the ability of cell homogenates to catalyze the formation of γ-glutamyl-α-amino[¹⁴C]butyrate from L-glutamate and L-α-amino[¹⁴C]butyrate was tested (L-α-aminobutyrate is a L-cysteine analog and is often used to replace L-cysteine in assays of γ-GCS activity). For comparison, cell homogenates of a wild-type strain of E. coli (JM105 E. coli), which is capable of GSH synthesis, and a strain of E. coli in which the γ-GCS gene was knocked out (gshA⁻ JM105 E. coli) were also tested. These were used as positive and negative controls, respectively. The amount of γ-glutamyl-α-amino[¹⁴C]butyrate formed (i.e., nmol γ-glutamyl-α-amino[¹⁴C]butyrate per mg of total protein) was plotted as a function of time (in minutes). As shown in FIG. 4, crude cell homogenates of S. agalactiae catalyzed a linear increase of product (i.e., γ-glutamyl-α-amino[¹⁴C]butyrate) over time, confirming that S. agalactiae has γ-GCS activity. The rate at which crude homogenates of S. agalactiae formed L-γ-glutamyl-L-α-amino[¹⁴C]butyrate was substantially less than seen with the native E. coli strain (JM105). As expected, the gshA⁻ strain of E. coli did not synthesize γ-glutamyl-α-amino[¹⁴C]butyrate. This example showed that GSH synthesis in S. agalactiae proceeds though the same initial step as in E. coli (i.e., the γ-GCS reaction) and not through some alternative pathway. The observation that S. agalactiae have lower γ-GCS activity than E. coli but maintain much higher GSH levels showed that the activity catalyzing GSH synthesis in S. agalactiae would be highly efficient in making GSH even when GSH levels were high.

### Example 2

This example demonstrated the isolation of γ-GCS activity, catalyzed by γ-GCS-GS, from S. agalactiae and the use of that protein to identify the gene encoding γ-GCS-GS.

Endogenous S. agalactiae γ-GCS activity was isolated and partially purified from 12 L of S. agalactiae cultures. S. agalactiae were grown for about 8 hrs (OD₆₀₀ of about 1.2), and then were harvested by centrifugation (yield: about 50 gram wet cell mass) and were frozen at −80° C. to facilitate cell breakage. The cells were then thawed, resuspended in isolation buffer (50 mM Tris HCl buffer, pH 7.4, 5 mM L-glutamate, 5 mM MgCl₂, and 1 mM dithiothreitol (DTT)), and broken by passage through a French pressure cell. The crude homogenate was clarified by centrifugation, and the supernatant solution was applied to a 2.5×20 cm (diameter×length) column of Whatman DE-52 anion exchange resin equilibrated with the isolation buffer. After washing with isolation buffer until OD₂₈₀ was about zero, S. agalactiae γ-GCS-GS was eluted with a linear gradient established between 400 ml of isolation buffer and 400 ml of isolation buffer containing 0.3 M NaCl. Fractions containing γ-GCS activity, as determined by an ADP formation assay (see below), were pooled, made 5 mM in MnCl₂, and applied to a 1×8 cm column of ATP affinity resin (C8-linked, 9 atom spacer; Sigma catalogue #A2767) that was equilibrated with isolation buffer that contained 5 mM MnCl₂ instead of 5 mM MgCl₂. The column was washed successively with about 50 ml equilibration buffer and about 25 ml of the original Mg²⁺-containing isolation buffer. S. agalactiae γ-GCS-GS was then eluted with 25 ml of the same buffer supplemented with 1 mM ATP. Fractions that contained γ-GCS activity were pooled and dialyzed against 8 L of 20 mM HEPES buffer, pH 7.8, containing 1 mM EDTA.

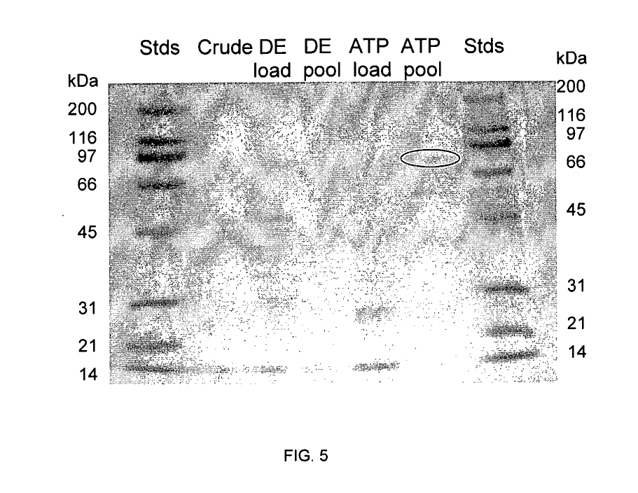

As described above, a protein with γ-GCS activity was isolated and purified from homogenates of S. agalactiae by centrifugation and sequential chromatography on DEAE-cellulose (i.e., DE-52 anion exchange resin) and ATP affinity resin. For the best preparation, the isolated protein exhibited two major bands (85 and 55 kDa) on Coomasie Blue-stained SDS-PAGE gel (FIG. 5), the Coomasie Blue-stained SDS-PAGE gel having been loaded in the first and last lanes with molecular weight markers (represented as ‘Stds’), in the second lane with crude homogenate of S. agalactiae (represented as ‘Crude’), in the third lane with DEAE cellulose column load (represented as ‘DE load’), in the fourth lane with DEAE cellulose column pool (represented as ‘DE pool’), in the fifth lane with ATP column load (represented as ‘ATP load’), and in the sixth lane with ATP column pool (represented as ‘ATP pool’). Both major protein bands from the sixth gel lane were subjected to in-gel trypsin digestion and MALDI-TOF analysis of the resulting peptide fragments. The circled band at about 85 kDa (FIG. 5) was identified by MALDI-TOF analysis as having an amino acid sequence consistent with the SAG1821 gene of S. agalactiae with a confidence interval of 2.50 (99%) as determined by Pro-found (Ver. 4.10.5, The Rockefeller University). The SAG1821 gene comprises a 2250 bp open-reading frame (ORF) and encodes a 750 amino acid protein. In the annotation of the S. agalactiae genome, SAG1821 was identified as coding a putative glutamate-cysteine ligase/amino acid ligase. Further analysis by the present inventors showed that the SAG1821 sequence encodes a of 85 kDa protein (750 amino acids) in which the N-terminal 518 amino acids (about 56 kDa) showed about 32% identity (43% similarity) with E. coli γ-GCS (about 56 kDa) and the C-terminal 390 amino acids in the sequence (about 40 kDa) showed about 24% identity (38% similarity) to E. coli D-Ala, D-Ala ligase (FIG. 1A and FIG. 1C).

Table 1 below shows the total γ-GCS activity and specific γ-GCS activity for various steps in the purification of the endogenous γ-GCS-GS of S. agalactiae. In Table 1, a unit was defined as the amount of enzyme activity required to catalyze the formation of 1 μmol of product per hour. Specific activity was shown as units per mg protein. Activity was determined at 37° C. based on ADP formation in reaction mixtures containing L-glutamate and L-cysteine and using pyruvate kinase and lactate dehydrogenase to couple ADP formation to NADH oxidation, which was monitored at 340 nm. Background formation of ADP, which was small, was determined in reaction mixtures lacking L-cysteine and was subtracted.

TABLE 1

| Purification | [Protein] | Volume | Total γ-GCS Activity | Specific γ-GCS | Yield |
|---|---|---|---|---|---|
| Step | (mg/ml) | (ml) | (units) | Activity (units/mg) | (%) |
| DEAE load | 5.4 | 34 | 730 | 4 | 100 |
| DEAE-52 | 0.039 | 153 | 413 | 69 | 57 |
| pool | | | | | |
| ATP load | 0.27 | 8 | 239 | 111 | 33 |
| ATP pool | 0.0018 | 12 | 67 | 3102 | 9 |

### Example 3

This example described the cloning of the S. agalactiae γ-GCS-GS gene, and the expression and purification of the protein. The putative S. agalactiae γ-GCS gene was cloned into a Qiagen pQE30 His₈-tag expression vector, and the protein was expressed in E. coli and purified to near homogeneity (about 98% pure).

Genomic DNA was isolated from S. agalactiae as described by M. G. Caparon and J. R. Scott (Meth. Enzymol. 204, 556-586 (1991)), and the putative γ-GCS-GS gene (SAG1821, now renamed gshAB) was isolated by PCR using a nested primer approach. Accordingly, a fragment containing SAG1821 and about 100 base pair flanking sequences was amplified using 5′ GATTAATAAGATTGGACTCAAAAG 3′ and 5′ ATTATGAGAATTTGGAATAGCG 3′ as primers. The PCR product was then inserted into a TOPO cloning vector, pCR2.1. Accuracy of the resulting plasmid was confirmed by DNA sequencing, and it was then used as a template in a second PCR step in which primers 5′ CGCGAGATCTCATGATTATCG 3′ and 5′ CGCGCTGCAGCCTAGCCTAAGGAAC 3′ were used to introduce unique Bgl II and Pst I restriction sites (boldly marked in the sequences) at the 5′ and 3′ ends, respectively. The amplified fragment was cut and introduced into the pQE30 expression vector immediately downstream of the His₈-tag site. The insert and flanking regions were sequenced to confirm that the vector insert matched the sequence reported for SAG1821 and thus coded for the native protein with an N-terminal His₈-tag.

To facilitate expression of large amounts of protein (i.e., over-expression), S. agalactiae γ-GCS-GS was expressed in SG13009 E. coli cells that were transformed with either pREP4 plasmid (used to prevent β-D-thiogalactopyranoside (IPTG)-independent expression) or with pRARE plasmid (used to code for rare tRNAs not otherwise plentiful in E. coli) in addition to the gshAB-bearing pQE30 plasmid. The pQE30, pREP4 and pRARE plasmids also code for ampicillin-, kanamycin- and chloramphenicol-resistance, respectively.

Transformed cells were grown, induced, and harvested by centrifugation, and were then broken using a French pressure cell. Crude homogenates were clarified by high-speed centrifugation, and γ-GCS activity was purified by chromatography on a column (2×8 cm) of Ni²⁺-NTA affinity resin. The column was equilibrated with 50 mM Tris HCl buffer, pH 7.4, containing 5 mM L-glutamate, 5 mM MgCl₂, and 5 mM β-mercaptoethanol, and the high-speed supernatant was loaded. After washing the column with equilibration buffer to remove proteins lacking a His-tag, the expressed His₈-tagged protein was eluted using the same buffer supplemented with 200 mM imidazole. Amount of γ-GCS activity was determined as described in Example 2. Amount of GS activity was determined similarly except the γ-GCS substrates (L-glutamate and L-cysteine) were replaced by the GS substrates (L-γ-glutamyl-L-cysteine and glycine). A summary of a typical purification in which expression was carried out using the pRARE auxiliary plasmid is shown in Table 2. It was observed that the purified enzyme catalyzed both the γ-GCS and the GS reactions. Since the GS-specific activity was higher than the specific activity for any known GS enzyme, it was apparent that the expressed 85 kDa S. agalactiae protein (rather than a trace impurity), accounted for the observed GS activity. Using the pREP4 auxiliary plasmid instead of the pRARE plasmid gave somewhat lower yields (325 mg total protein vs. 442 mg protein using pRARE) but the specific activities of the γ-GCS activity (870 units/mg) and GS activities (1143 units/mg) were similar. These results and the data in Table 2 showed that for S. agalactiae γ-GCS-GS the ratio of γ-GCS to GS specific activity was about 0.77. The fact that GS activity is higher than γ-GCS activity helps prevent large amounts of the L-γ-glutamyl-L-cysteine intermediate from accumulating during GSH synthesis, and is one advantage in using S. agalactiae γ-GCS-GS for GSH synthesis.

TABLE 2

| | | | Total | Specific | | |
|---|---|---|---|---|---|---|
| | Protein | | γ-GCS | activity | Total GS | Specific |
| Purification | Conc. | Volume | activity | γ-GCS | activity | activity GS |
| Step | (mg/ml) | (ml) | (units) | (units/mg) | (units) | (units/mg) |
| Ni²⁺-NTA | 14.4 | 116 | not determined | not determined | not determined | not determined |
| load | | | | | | |
| Ni²⁺-NTA | 20.1 | 22 | 406,219 | 919 | 513,117 | 1,160 |
| pool | | | | | | |

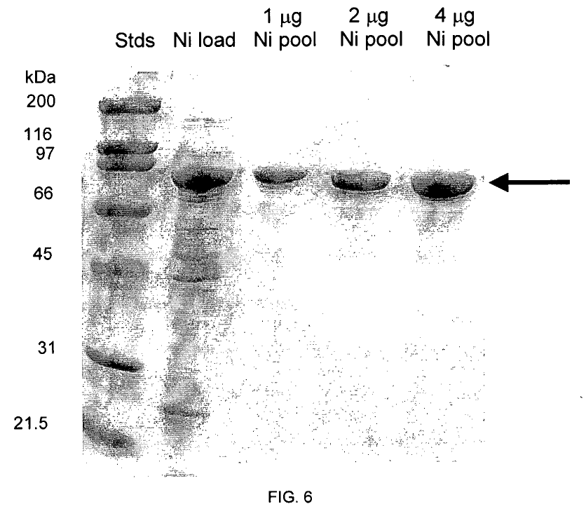

The final preparations of S. agalactiae γ-GCS-GS were highly pure with respect to the expected about 85 kDa protein as shown on Coomasie-blue stained SDS-PAGE gel (FIG. 6), the Coomasie-blue stained SDS-PAGE gel having been loaded in the first and last lanes with molecular weight markers (represented as ‘Stds’), in the second lane with Ni²⁺-NTA column load (represented as ‘Ni-load’), in the third lane with 1 μg of Ni²⁺-NTA column pool (represented as ‘1 μg-Ni pool’), in the fourth lane with 2 μg Ni²⁺-NTA column pool (represented as ‘2 μg-Ni pool’), and in the fifth lane with 4 μg Ni²⁺-NTA column pool (represented as ‘4 μg-Ni pool”). In-gel trypsin digestion and MALDI-TOF analysis of the fragments established that the band at approximately 85 kDa corresponded to full-length S. agalactiae γ-GCS-GS with an N-terminal His₈-tag (the expected molecular mass with His₈-tag is 88 kDa), whereas the two most visible trace impurity bands (molecular masses of 70 and 55 kDa) were shown by MALDI-TOF analysis to correspond to His₈-tagged N-terminal fragments of S. agalactiae γ-GCS-GS. Scanning of the stained gel indicated that the major band at approximately 85 kDa accounted for about 98% of the protein in the final preparations. The full length of S. agalactiae γ-GCS-GS band is indicated by the arrow in FIG. 6.

### Example 4

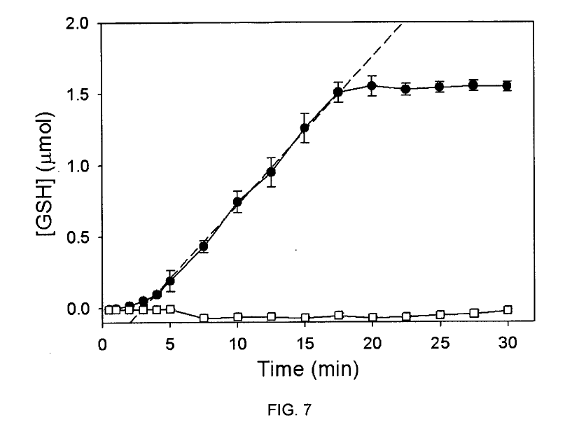

This example demonstrated that purified S. agalactiae γ-GCS-GS in the presence of ATP catalyzed the formation of GSH from its constituent amino acids, (100 mM L-glutamate, 2 mM L-cysteine and 50 mM glycine). The formation of GSH was monitored using the GSSG reductase-dependent enzymatic recycling assay described in Example 1 (modified Tietze assay), and the results were plotted as a function of time (FIG. 7).

As shown in FIG. 7, GSH synthesis proceeded linearly after an initial lag that was attributed to the need to accumulate sufficient L-γ-glutamyl-L-cysteine for efficient GS reaction. The attenuation of GSH formation after about twenty minutes was attributed to L-cysteine depletion; its concentration is diminished by both enzymatic use and oxidation to cystine in this example. In the absence of glycine, synthesis of γ-glutamylcysteine, rather than GSH, occurred. Thus, under conditions similar to those used in the FIG. 7 studies, incubation of purified γ-GCS-GS with ATP, L-glutamate and L-[³⁵S]cysteine yielded L-γ-glutamyl-L-[³⁵S]cysteine as determined using small columns of Dowex 1 to separate unreacted L-[³⁵S]cyst(e)ine from L-γ-glutamyl-L-[³⁵S]cysteine (not shown).

### Example 5

This example demonstrated the characterization of the γ-GCS activity of S. agalactiae γ-GCS-GS with respect to its affinity for its substrates (Kₘ values) and the reaction velocity attained when substrates were present at saturating levels (Vₘₐₓ values). Table 3 compares γ-GCS Kₘ and Vₘₐₓ values among S. agalactiae γ-GCS-GS, E. coli γ-GCS, human γ-GCS, and human γ-GCSc (catalytic subunit only) for different substrates (L-glutamate, L-cysteine, L-α-aminobutyrate, and ATP). Results for E. coli γ-GCS are from B. S. Kelly et al. J. Biol. Chem. 277, 50-58 (2002). Results for human enzymes are from I. Misra and O. W. Griffith, Prot. Exp. Purif. 13, 268-276 (1998).

TABLE 3

| | Kinetic | S. agalactiae | E. coli | Human | Human |
|---|---|---|---|---|---|
| Substrate | Value | γ-GCS-GS | γ-GCS | γ-GCS | γ-GCSc |
| L-glutamate | Kₘ | 23 ± 2 mM | 1.9 ± 0.2 mM | 1.9 ± 0.2 mM | 3.2 ± 0.1 mM |
| | Vₘₐₓ | 1341 ± 145 units/mg | 3170 ± 125 units/mg | ~1500 units/mg | ~1250 units/mg |
| L-cysteine | Kₘ | 160 ± 9 μM | 100 ± 20 μM | 100 ± 20 μM | 130 ± 10 μM |
| | Vₘₐₓ | 1126 ± 21 units/mg | 3590 ± 200 units/mg | ~1500 units/mg | ~1250 units/mg |
| L-α-amino- | Kₘ | 8.3 ± 1 mM | 3.9 ± 0.4 mM | 1.3 mM | 1.7 mM |
| butyrate | Vₘₐₓ | 1148 ± 73 units/mg | 2970 ± 120 units/mg | ~1500 units/mg | ~1250 units/mg |
| ATP | Kₘ | 66 ± 9 μM | 62 μM | 400 ± 40 μM | not determined |
| | Vₘₐₓ | 1332 ± 120 units/mg | ~3000 units/mg | ~1500 units/mg | not determined |

As shown in Table 3, the γ-GCS specific activity of S. agalactiae γ-GCS-GS, determined under Vₘₐₓ conditions (about 1300 units/mg), was in the range of human γ-GCS and about one half that of E. coli γ-GCS. Also shown in Table 3 are the substrate Kₘ values determined for the γ-GCS activity of S. agalactiae γ-GCS-GS and, for comparison, the Kₘ values determined for those substrates with known monofunctional γ-GCS-enzymes. The measured Kₘ values for ATP and L-cysteine were similar to those reported for E. coli. In contrast, the Kₘ values for L-glutamate and L-α-aminobutyrate were about ten-fold and about two-fold higher, respectively, in S. agalactiae γ-GCS-GS. In Table 3, a unit was defined as the amount of enzyme activity needed to form one μmol of product per hour.

### Example 6

This example demonstrated the characterization of the GS activity of S. agalactiae γ-GCS-GS with respect to its affinity for its substrates (Kₘ values) and the reaction velocities attained when substrates were present at saturating levels (Vₘₐₓ values). Table 4 compares GS activity Kₘ and Vₘₐₓ values determined for S. agalactiae γ-GCS-GS, E. coli GS, rat GS, and human GS for different substrates (L-γ-glutamyl-L-cysteine, L-γ-glutamyl-L-α-aminobutyrate, glycine, and ATP). Results for E. coli GS are from H. Gushima et al. J. Appl. Biochem. 5, 210-218 (1983). Results for rat GS are from J. L. Luo et al. Biochem. Biophys. Res. Commun. 275, 577-581 (2000). Results for human GS are from R. Njalsson et al. Biochem. Biophys. Res. Commun. 289, 80-84 (2001) and R. Njalsson et al. Biochem. J. 349, 275-279 (2000).

TABLE 4

| | Kinetic | S. agalactiae | E. coli | Rat | Human |
|---|---|---|---|---|---|
| Substrate | Value | γ-GCS-GS | GS | GS | GS |
| L-γ- | Kₘ | 3.9 ± 0.5 mM | 2.6 mM | not determined | not determined |
| glutamyl- | Vₘₐₓ | 2264 ± 896 units/mg | 650 units/mg | not determined | not determined |
| L-cysteine | | | | | |
| L-γ- | Kₘ | 11.5 ± 1.5 mM | not determined | 42 μM | 63-164 μM |
| glutamyl- | Vₘₐₓ | 2648 ± 597 units/mg | not determined | 678 units/mg | 336 units/mg |
| L-α-amino- | | | | | |
| butyrate | | | | | |
| glycine | Kₘ | 6 ± 0.5 mM | 2.0 mM | 913 μM | 1.3 ± 0.3 mM |
| | Vₘₐₓ | 2326 ± 584 units/mg | 650 units/mg | 678 units/mg | 361 ± 84 units/mg |
| ATP | Kₘ | 420 ± 49 μM | 1.8 mM | 37 μM | 220 ± 30 μM |
| | Vₘₐₓ | 1662 ± 77 units/mg | 650 units/mg | 678 units/mg | 361 ± 84 units/mg |

As shown in Table 4, the GS-specific activity of S. agalactiae γ-GCS-GS, determined under Vₘₐₓ conditions (about 2000 units/mg), was about six-fold higher than that of human GS and about three-fold higher than that of E. coli GS. Also shown in Table 4 are the substrate Kₘ values determined for the GS activity of S. agalactiae γ-GCS-GS and, for comparison, the Kₘ values determined for those substrates with known monofunctional GS enzymes. As shown, the Kₘ values for L-γ-glutamyl-L-cysteine, L-γ-glutamyl-L-α-aminobutyrate, and glycine were found to be 2- to 600-fold higher in S. agalactiae than in the other species. The Kₘ value of ATP was found to be substantially lower than the value seen with E. coli, but was 2-fold higher than reported for human GS. In Table 4, a unit was defined as the amount of enzyme activity needed to form one μmol of product per hour.

### Example 7

This example demonstrated that S. agalactiae γ-GCS-GS was not inhibited by GSH or cystamine but was inhibited by BSO. The data are shown in Table 5. Results for E. coli γ-GCS are from B. S. Kelly et al. J. Biol. Chem. 277, 50-58 (2002). Results for human γ-GCS are from I. Misra and O. W. Griffith, Prot. Exp. Purif. 13, 268-276 (1998) and F. Tietze, Anal. Biochem. 27, 502-522 (1969).

TABLE 5

| | Kinetic | S. agalactiae | E. coli | Human |
|---|---|---|---|---|
| Inhibitor | Value | γ-GCS-GS | γ-GCS | γ-GCS |
| Glutathione | Kᵢ | no inhibition | 2.7 mM | 5.8 mM |
| (GSH) | | (<5% at 100 mM) | | |
| Cystamine | Kᵢ | no inhibition | no inhibition | 2 mM |
| L-Buthionine- | Kᵢ | 4.9 ± 0.2 mM | 66 μM | 25 μM |
| S-sulfoximine | K_{d} | 2.8 ± 0.3 mM | 66 μM | 25 μM |
| (L-S-BSO) | k_{inact} | 0.13 ± 0.01/min | ~0.30/min | 3.9/min |
| | t_{1/2} | 5.5 ± 0.5 min | 2.3 min | 11 sec |

Inhibition of γ-GCS activity by GSH or cystamine was determined in reaction mixtures containing 25 mM L-glutamate, 1 mM L-cysteine, 0 to 100 mM GSH or cystamine and other buffers and reactants required for carrying out the standard assay based on ADP formation as described in Example 2. As shown in Table 5, GSH at concentrations up to 100 mM caused <5% inhibition, indicating that the Kᵢ for GSH would be >>100 mM in experiments of the type described here where non-varied substrates are present at concentrations ranging from their Kₘ values to 10 times their Kₘ values. Cystamine at concentrations up to 100 mM caused no reproducibly measurable inhibition.

The initial binding of L-S—BSO to S. agalactiae γ-GCS-GS (i.e., reversible, competitive inhibition by L-S—BSO) was determined in terms of the observed Kᵢ, which was measured using the standard γ-GCS activity assay for ADP formation with L-glutamate concentrations ranging from 17-100 mM and L-S—BSO concentrations ranging from 0-10 mM. To characterize irreversible inhibition by L-S—BSO (i.e., mechanism-based inactivation), k_{inact} (rate constant for inactivation), K_{D} (initial binding equilibrium constant), and the t_{1/2} (half-life) for inactivation were determined. For these studies, S. agalactiae γ-GCS-GS was preincubated in 500 μl reaction mixtures containing 210 mM Tris HCl buffer, pH 8.2, 0.4 mM EDTA, 140 mM KCl, 10 mM ATP, 35 mM MgCl₂ and varying amounts of L-S—BSO (˜0.62 mM to ˜3.1 mM) at 37° C. At set time points ranging from one to 30 minutes, 5 μl aliquots were removed and assayed for residual enzyme activity using the standard γ-GCS assay based on ADP formation. Inhibition progress curves were plotted for each concentration of L-S—BSO (log of percent remaining activity vs. time), and the apparent k_{inact} values were calculated from the slopes of those lines. Reciprocals of the apparent k_{inact} values were replotted against 1/[L-S—BSO] to establish 1/k_{inact} (Y-intercept) and −1/K_{D}, (X-intercept). The values determined for Kᵢ, K_{d}, K_{inact}, and t_{1/2} are shown in Table 5.

In studies similar to those described for BSO the initial binding constant (Kᵢ) for D,L-2-amino-4-phosphonobutyric acid was found to be 13 mM and the Kᵢ for glufosinate (ammonium salt) was found to be 29 mM. L-Methionine-S,R-sulfoximine and D,L-2-amino-5-phosphonovaleric acid were not effective inhibitors (Kᵢ>100 mM).

### Example 8

This example described the cloning of the E. faecalis γ-GCS-GS gene (E. faecalis gshAB) and the expression, purification and characterization of the protein. The E. faecalis gshAB was cloned into the pQE30T expression vector immediately downstream of the His₆-tag and tobacco etch virus (TEV) protease cleavage sites and the protein was expressed in E. coli and purified to near homogeneity.

Genomic DNA was isolated from E. faecalis EF3089. The desired gshAB was amplified directly using primers (5′ CGCGGGATCCATGAATTATAGAGAATTAATGCAAAAGAAAAATGTTCG 3′ and 5′ CGCGAAGCTTTTATTGAACCACTTCTGGGTATAAAAGTTTTAAAACG 3′) that introduced unique Bam H1 and Hind III restriction sites (underlined) at the 5′ and 3′ ends, respectively. The amplified fragment was cut and introduced into the pQE30T expression vector immediately downstream of the His₆-tag and tobacco etch virus (TEV) protease cleavage sites, and the insert and flanking regions were sequenced to confirm that the vector insert matched the sequence reported for EF3089 (now known to be gshAB) in the completed E. faecalis genome. The His₆-tag and TEV linker added the sequence MRGSHHHHHHGSENLYFQGS onto the N-terminal end of the native E. faecalis sequence shown as SEQ ID NO:12 in the Sequence Listing Section; TEV protease cleaves the linker between Q and G.

E. faecalis γ-GCS-GS was expressed and purified to near homogeneity using the same procedures used to express and purify S. agalactiae γ-GCS-GS (see Example 3) except that 50 mM L-Glu was added to the isolation buffer. The purified enzyme had γ-GCS specific activity of 240 units/mg and a GS specific activity of 2297 units/mg. The ratio of activities was about 0.23, significantly lower than observed with S. agalactiae γ-GCS-GS.

The kinetic constants (Kₘ values) for the γ-GCS and GS activities of E. faecalis γ-GCS-GS were determined using the same methods described for S. agalactiae γ-GCS-GS (see Examples 4 and 5). Results were as follows: For γ-GCS activity: L-glutamate Kₘ, 79±14 mM; L-cysteine Kₘ, 192±14 μM; ATP, Kₘ, 2.3±0.1 mM. For GS activity: L-γ-glutamyl-L-cysteine Kₘ, 4.30±0.05 mM; glycine, Kₘ, 5.0±0.5 mM; ATP, Kₘ, 179±5 μM. Inhibition by GSH was tested in reaction mixtures similar to those used with S. agalactiae γ-GCS-GS, containing 10 mM to 100 mM L-glutamate, 1 mM L-cysteine, 10 mM ATP and GSH from 0 to 100 mM. The results were plotted according to Lineweaver and Burke. GSH was an effective inhibitor, competitive with L-glutamate, and exhibited a Kᵢ value of 25.1±0.8 mM.

### Example 9

This example described the cloning of the S. mutans γ-GCS-GS gene (S. mutans gshAB) and the expression, purification and characterization of the protein. The S. mutans gshAB was cloned into the pQE30T expression vector immediately downstream of the His₆-tag and TEV protease cleavage sites and the protein was expressed in E. coli and purified to about 10% purity.

Cloning and expression of the γ-GCS-GS of S. mutans SMU.267c was carried out similarly to the procedure used for E. faecalis γ-GCS-GS except the primers were 5′ CGCGAGATCTATGCACTCAAATCAATTATTACAGCATGC 3′ and 5′ CGCGAAGCTTTTATATCTTGGTGCTTATTTCAGGAAAGAGC 3′ and introduced unique Bgl II and Hind III sites (underlined), respectively. Expression yielded less soluble protein than seen with S. agalactiae and E. faecalis γ-GCS-GS, but the enzyme was nonetheless isolated in about 10% purity as judged by Coomasie blue stained SDS-PAGE gels. Although both γ-GCS and GS activities were determined to be present, only the γ-GCS activity has been characterized to date in terms of substrate affinities. The determined Kₘ values for L-glutamate, L-cysteine and ATP were 17±1 mM, 144±4 μM and 288±105 μM, respectively. Inhibition by GSH was competitive with L-glutamate and characterized by a Kᵢ of 67±0.8 mM. It was apparent that the S. mutans enzyme has a sensitivity to GSH inhibition that was intermediate between that of the S. agalactiae and E. faecalis enzymes.

### Example 10 (Prophetic)

This example describes the use of γ-GCS-GS to synthesize isotopically labeled GSH in vitro. The same general procedure can be used to synthesize GSH analogs in which the L-glutamate, L-cysteine or glycine moieties are replaced by analogs of those amino acids.

A reaction mixture is prepared containing in a final volume of 25 ml the following: 100 mM Tris HCl buffer, pH 8.2, 50 mM KCl, 50 mM L-glutamate, 50 mM L-cysteine, 55 mM [¹⁴C]glycine (100 mCi), 20 mM ATP, 150 mM phospho-enol-pyruvate (PEP), 150 mM MgCl₂, 1 mM EDTA, 10 mM dithiothreitol, 10 IU of pyruvate kinase, and 2000 units of S. agalactiae γ-GCS-GS. The reaction mixture is mixed and placed in a 30° C. water bath. At 30 min intervals 10 μl portions are removed, diluted into 1 ml of 20 mM acetic acid, which stops the reaction, and that solution is applied to a small columns (0.5×5 cm) of Dowex 1 acetate. The resin is washed with 10 ml of 20 mM acetic acid, which elutes unreacted [¹⁴C]glycine. The resin is then washed with 4 ml of 2 M, which elutes [¹⁴C-glycine]GSH. That product and the [¹⁴C]glycine eluted earlier are separately quantitated by liquid scintillation counting and percent completion is calculated as 100×[¹⁴C]GSH/([¹⁴C]GSH+[¹⁴C]glycine). After 2 hrs the reaction is >50% complete and after incubation overnight, it is >85% complete. As an alternative, formation of GSH product can be followed using the Tiezte assay described in Example 1 or formation of GSH analogs can be followed by HPLC using any of several well-established HPLC systems used to quantitate GSH and its analogs. If necessary more γ-GCS-GS, ATP and/or PEP is added to achieve at least 80% incorporation of the most limiting amino acid (L-cysteine in this case) into GSH.

Once the reaction mixture meets or surpasses the completion criteria, 5′-sulfosalicyclic acid is added to a final concentration of 5%, and the solution is centrifuged to remove precipitated protein. The supernatant solution is then applied to a column (1.5×10 cm) of Dowex 50×8 (200-400 mesh, H⁺ form), and the resin is washed with 100 ml of water to remove Cl⁻, ATP, PEP, EDTA and other anionic or uncharged species. [¹⁴C]GSH and residual amino acids are then eluted with 1 M pyridine. Fractions containing ¹⁴C (or in the case of non-radioactive syntheses, fractions containing thiol (detected with 5,5′-dithiobis(2-nitrobenzoic acid (DTNB)) or fractions giving a positive ninhydrin reaction) are pooled and concentrated to dryness under vacuum using a rotary evaporator and a bath temperature of 20 to 40° C. The residue is then dissolved in 50 ml of water and applied to a column (1.5×20 cm) of Dowex 1×8 (200-400 mesh, acetate form). The resin is washed with 100 ml of 0.1 M acetic acid, which completes removal of residual L-cysteine and [¹⁴C]glycine, and then with 0.6 M acetic acid, which removes residual L-glutamate. [¹⁴C]GSH is then eluted using 1.2 M acetic acid. Fractions containing GSH, detected by any of the methods listed above, are pooled and concentrated under vacuum by rotary evaporation. Water is added and removed twice to assure removal of acetic acid, and the residual solid is crystallized from ethanol/water. The yield is about 300 mg (1 mmol) of [¹⁴C-glycine]GSH. In the event that the final product contains L-γ-glutamyl-L-cysteine, a solution of the product is treated with purified rat kidney γ-glutamylcyclotransferase to convert the contaminant to 5-oxoproline and cysteine, and the chromatography on Dowex 1 is repeated.

By procedures similar to that described above GSH is synthesized containing [¹⁴C]glutamate, or [³⁵S]cysteine, or [¹³C]cysteine, or [¹⁵N]glycine, or both [¹³C]cysteine and [¹⁵N]glycine, or [¹⁴C]glutamate, [¹³]cysteine and [¹⁵N]glycine. Analogs of GSH are prepared similarly by replacing L-glutamate, L-cysteine and/or glycine in the reaction mixture with analogs of those amino acids that are recognized as substrates by γ-GCS-GS. In this manner, the analog of GSH in which L-cysteine is replaced by L-α-aminobutyrate, a GSH analog known as opthalmic acid, is prepared in >75% isolated yield. L-cysteine may alternatively be replaced by L-β-chloroalanine, L-β-cyanoalanine, L-serine and L-allo-threonine. L-Glutamate may be replaced by N-methyl-L-glutamate. These compounds are useful for determining the specificity of transporters and enzymes that normally use GSH as a substrate. Use of γ-GCS-GS to carry out the synthesis avoids the need to purify two separate enzymes (i.e., γ-GCS and GS) and, in the case of S. agalactiae γ-GCS-GS avoids the problem of GSH causing inhibition of the γ-GCS reaction as it accumulates in the reaction mixture. Enzymatic synthesis of GSH or its analogs is particularly advantageous when incorporation of hazardous (e.g., radioactive), expensive or rare amino acids (e.g., [¹³C-carboxyl]cysteine) is required because it avoids the need to synthesize the protected amino acids or deprotect the product. Those steps are required for chemical syntheses.

### Example 11

This example illustrated the use of gshAB and γ-GCS-GS to cause the synthesis of GSH in an organism otherwise unable to synthesize GSH. In the case exemplified a plasmid bearing gshAB was inserted into an E. coli strain in which the gene coding for γ-GCS (i.e., gshA) was previously knocked out.

Glutathione-deficient JM105 E. coli in which gshA was knocked out (i.e., gshA⁻ JM105) were co-transformed with a pQE30 plasmid bearing S. agalactiae gshAB and a pREP4 plasmid. A fifty-ml starter culture was initiated by inoculating rich medium (2×YT medium) containing kanamycin and ampicillin with a single colony of the transformed cells. After growing that culture overnight, a liter culture was initiated by inoculating 2×YT medium supplemented with kanamycin and ampicillin with 10 ml of the starter culture. Expression of S. agalactiae γ-GCS-GS was induced by the addition of 1 mM IPTG when the OD₆₀₀ of the culture was 0.6. At the time of induction, the growth temperature was reduced from 37° C. to 25° C., and the culture was grown for an additional 24 hrs. The cells were harvested, washed 3 times with PBS, and broken by passage through a French Pressure Cell. Aliquots of the clarified supernatant were acidified with 5′-sulfosalicyclic acid to a final concentration of 5% to precipitate the protein. The precipitated protein was discarded and the resulting supernatant was assayed for total GSH using the GSSG reductase-dependent GSSG to GSH recycling assay described earlier (see Example 1).

Total GSH levels in the gshA⁻ E. coli cells transformed with pQE30 plasmid containing S. agalactiae gshAB was 6.5±0.7 nmol GSH/mg protein. Total GSH levels in gshA⁻ E. coli that were grown similarly but were not transformed with pQE30 plasmid were too low to be detected. The experiment demonstrates that transformation with a plasmid bearing gshAB can cause GSH synthesis in cells not able to make GSH.

### Example 12 (Prophetic)

In an experiment similar to that in Example 11 gshA⁻, gshB⁻ E. coli (i.e., E. coli in which both γ-GCS and GS were knocked out) are transformed with the same plasmid as used in Example 11 and the cells are grown 36 hrs to late log phase in medium supplemented with 10 mM each of L-glutamate, L-cysteine and glycine. Total GSH levels are 35 nmol/mg protein, nearly two-fold the level seen in wild-type E. coli.

This example illustrates that total levels of GSH attained in cells transformed using the S. agalactiae gshAB gene can be increased by continuing the cell growth longer and by providing additional L-glutamate, L-cysteine and glycine in the growth medium. Total GSH levels can also be increased by transforming the cells with a plasmid that does not require IPTG induction, or by transforming using a plasmid that causes incorporation of gshAB into the genome of the transformed organism downstream of a housekeeping gene (i.e., into the genome in a position where γ-GCS-GS is continuously expressed). This last approach, carried out using a method selected from methods known to work for cells of the type being transformed, results in a stably transfected organism that maintains an intracellular GSH concentration of at least 20 nmol/mg protein.

A similar approach can be used to cause GSH synthesis in prokaryotic or eukaryotic organisms that naturally lack GSH synthesis or that have less capacity for GSH synthesis than is desired. Alternatively, with both prokaryotic and eukaryotic cells it is possible to provide gshAB on a plasmid that causes gshAB to be stably incorporated into the genome of the treated cell. In these manners it is possible to cause GSH synthesis in a wide variety of cell types, and such cells are rendered resistant to various toxicities, particularly toxicity due to oxidative stress, nitrosative stress, reactive electrophiles or heavy metals. Such cells may also provide a useful source of GSH (e.g., for use in nutritional supplements). Use of gshAB from S. agalactiae is particularly useful for these purposes because the protein expressed, S. agalactiae γ-GCS-GS, is not feedback inhibited by GSH and therefore causes synthesis of GSH to continue until high concentrations of GSH are attained.

### Example 13 (Prophetic)

This example describes a high throughput method for identifying inhibitors of γ-GCS-GS and for establishing their utility as potential anti-microbial agents. The method is useful for screening, for example, combinatorial libraries of possible inhibitors having structures related to the γ-GCS-GS substrates and therefore likely to be inhibitors. It is also useful for screening large commercially available libraries of random chemicals.

Possible inhibitors are first screened using isolated γ-GCS-GS that is prepared as described in Examples 2 or 3 from the species of interest. Specifically, in each well of a 96-well plate is placed 200 μl of a solution containing 100 mM Tris HCl buffer, pH 8.0, 25 mM L-glutamate, 0.1 mM L-cysteine, 5 mM glycine, 10 mM ATP, 20 mM MgCl₂, and 1.0 mM EDTA. Samples of possible inhibitors are added to individual wells. For example, 0, 1 mM and 10 mM concentrations of S-alkyl-L-homocysteine-S,R-sulfoximines having S-alkyl groups of 1 to 10 carbon atoms are added to individual wells (the 4 carbon S-alkyl group compound is L-S,R—BSO). Other wells receive 1 μg, 10 μg, or 100 μg samples taken from a combinatorial library containing derivatives of glutamate. To each well is then added 0.02 unit of S. agalactiae γ-GCS-GS, the plate is mixed and incubated for 1 hr at 37° C. At that time, a 10 μl portion of the solution in each well is transferred to the corresponding well of another 96-well plate in which each well additionally contains 100 μl of a solution containing 125 mM KPi, pH 7.4, 5 mM EDTA, 0.25 mM NADPH, and 0.6 mM DTNB. The plate is agitated to mix each well and 0.05 unit of commercial GSSG reductase is added to each well (one unit of GSSG reductase is defined as the amount of activity necessary to reduce 1 μmol of GSSG per minute). The plate is immediately put into a 96-well plate reader, and the increase in absorbance at 412 nm is monitored for 10 min. The assay works as follows: Any thiol in a well immediately reduces DTNB to the free thiol form (i.e., 5-thiol-2-nitrobenzoic acid (TNB)), which is yellow and detected at 412 nm. If the thiol is GSH (i.e., product formed by γ-GCS-GS), then the co-product formed in the reduction of DTNB is GSSG or the disulfide of GSH and TNB (GS-TNB). In a NADPH-dependent reaction GSSG reductase immediately reduces GSSG or GS-TNB back to GSH or GSH and TNB. GSH again reduces DTNB, increasing the yellow color. The rate of increase in yellow color, detected at 412 nm, is linearly proportional to the amount of GSH transferred from the well of the first plate. In the absence of inhibitor, the 10 μl sample taken from the first 96 well plate contains ˜0.5 nmol of GSH and gives an increase in A₄₁₂ of ˜1 OD/min when the second plate is in the reader (the recorded rate is based on the steepest part of the progress curve, ignoring, if necessary, later parts of the progress curve where A₄₁₂ is too great to accurately measure). If necessary, the amount of γ-GCS-GS used in the first reaction or the amount of GSSG reductase used in the second reaction are adjusted to achieve a rate between 0.2 and 1.2 OD/min. Compounds inhibiting γ-GCS-GS cause less GSH to be synthesized and are identified on the basis that the corresponding well in the second plate shows a lower rate of increase in A₄₁₂ compared to second plate wells corresponding to no inhibitor wells on the first plate. In the prophetic experiment described, 1 and 10 mM L-S—BSO causes >50% inhibition and one compound from the combinatorial library (N-methyl-L-glutamate) causes 10% inhibition when 100 μg is tested. The other compounds are less effective or not effective inhibitors. Inhibition by active compounds is confirmed, localized to either the γ-GCS activity or the GS activity, and is characterized in terms of Kᵢ value using the ADP formation assays as described in Examples 5, 6 and 7.

Compounds shown to inhibit γ-GCS-GS are screened in vivo for their ability to inhibit GSH synthesis in bacteria containing γ-GCS-GS as follows: In each well of a 96-well plate is placed 100 μl of a culture of the bacterial species of interest previously grown to an OD₆₀₀ of 0.01 in chemically defined, GSH-free media as described in Example 1.

To individual wells 0.1 ng to 100 μg samples of various γ-GCS-GS inhibitors identified as described above are immediately added. The plate is incubated overnight at 37° C., and then centrifuged to sediment the bacteria to the bottom of the wells. The supernatant is removed and the cells are resuspended in 100 μl of phosphate-buffered saline supplemented with 20 mg/ml lysozyme. That mixture is incubated for 1 hr at 37° C. to lyse the cells. To those solutions are added 100 μl of freshly prepared 125 mM KPi, pH 7.4, 5 mM EDTA (which prevents further γ-GCS-GS reaction), 0.25 mM NADPH, and 0.6 mM DTNB containing 50 units/ml of commercial GSSG reductase. Wells containing cells that are able to make GSH during the initial incubation period turn yellow at a rapid rate due to DTNB reduction caused by the GSSG to GSH recycling (see above). Wells containing bacteria in which γ-GCS-GS is completely inhibited turn yellow at a slow rate that is equal to the rate seen in control wells that do not contain bacteria. Partial inhibition, observed as intermediate rates of yellow color formation, is detected and quantitated using a plate reader. In an experiment S. agalactiae, wells containing 10 and 100 μg of L-S—BSO show ˜10% and >90% inhibition, respectively. Inhibition of cell growth is distinguished from inhibition of GSH synthesis per se by determining the OD₆₀₀ of the individual wells prior to cell lysis (i.e., bacteria grew more slowly or not at all in wells with lower OD₆₀₀ readings).

Bacteria that rely on γ-GCS-GS for GSH synthesis are more susceptible to oxidative stress (i.e., stress due to reactive oxygen species (ROS)) when their GSH pool is decreased by exposure to a γ-GCS-GS inhibitor. Relevant sources of oxidative stress are the immune system of an infected host (e.g., ROS made by macrophages and neutrophiles activated by infection) and certain redox cycling antibiotics such as nitrofurantoin. It is possible to demonstrate synergy between γ-GCS-GS inhibitors and oxidant stress increasing treatments using the 96-well plate assay described above by comparing bacterial growth in wells containing inhibitor alone, wells containing a source of oxidant stress alone, and wells containing both a γ-GCS-GS inhibitor and a source of oxidant stress (e.g., activated human neutrophils, which release several oxidants including hypochlorite, or glucose oxidase plus glucose, which forms hydrogen peroxide, or xanthine oxidase plus xanthine, which forms superoxide). Rate of bacterial growth is determined by monitoring OD₆₀₀ at hourly intervals for 24 hours. In this prophetic example, it is found that growth of E. faecalis is inhibited less than 5% by 10 μg of L-S—BSO alone, about 10% by 1 μg of nitrofurantoin alone, but about 30% by 10 μg of L-S—BSO plus 1 μg of nitrofurantoin.

### Example 14 (Prophetic)

This example illustrates the use of a γ-GCS-GS inhibitor to treat infection caused by a bacteria relying on γ-GCS-GS for synthesis of GSH.

A 45 year old woman weighing 50 kg presents to her physician with a urinary tract infection caused by E. faecalis. She is given one 500 mg capsule of L-S—BSO by mouth every 6 hours for two days without resolution of the infection. The dose is increased to two 500 mg capsules every 6 hours and at the end of the second day her urine culture is still positive for E. faecalis but by fourth day her urine is free of detectable bacteria. Two months later she returns with a recurrence of the urinary tract infection due to E. faecalis. She is treated with one 500 mg capsule of L-S—BSO and 50 mg of nitrofurantoin by mouth every 6 hours. At the end of the second day her urine is free of bacteria.

A 25 year-old woman who is allergic to penicillin is seen for prenatal screening prior to delivery of her first child. Routine screening shows a vaginal colonization with S. agalactiae (Group B Streptococcus) that is resistant to erythromycin. Since S. agalactiae infections commonly lead to infection of babies during parturition and sometimes cause fatal meningitis, the woman is instructed to take one 500 mg capsule of L-S—BSO by mouth every 6 hours beginning 2 days before her due date. She delivers on schedule and no S. agalactiae bacteria are detected in pre-delivery vaginal swabs. The baby is not infected.

The foregoing descriptions of specific embodiments of the present invention have been presented for purposes of illustration and description. They are not intended to be exhaustive or to limit the invention to the precise forms disclosed, and many modifications and variations are possible in light of the above teaching. The embodiments/examples are chosen and described in order to best explain the principles of the invention and its practical application, and, thereby, to enable others skilled in the art to best utilize the invention and various embodiments/examples with various modifications as are suited to the particular use contemplated. It is understood that various omissions, and substitutions of equivalents are contemplated as circumstance may suggest or render expedient.

1. An isolated DNA molecule encoding a bifunctional enzyme with γ-glutamylcysteine synthetase and glutathione synthetase activities.

2. An isolated DNA molecule as claimed in claim 1 which has a sequence identical to a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, and SEQ ID NO: 27.

3. An isolated DNA molecule encoding a protein which has at least 27% amino acid sequence indentity to SEQ ID NO: 2, which includes in its carboxyl-terminal section an ATP grasp domain, and which is functional to perform two enzymatic activities, γ-glutamylcysteine synthetase and glutathioine synthetase.

4. An isolated DNA molecule as claimed in claim 3 wherein the encoded protein includes, when aligned by sequence alignment with SEQ ID NO:2, residues identical to at least 50 of the the following 57 residues in SEQ ID NO; 2: G22, E24, R29, H42, P43, G47, T68, P69, P100, S102, R126, L129, Y133, G142, H144, L157, Y174, W186, L191, A194, Y237, R261, E280, R282, D285, L286, L428, S429, Q431, D448, K489, L492, P500,1536, E565, R574, F575, R588, A591, N592, G595, K608, N609, L613, R614, G615, P621, E631, L654, R655, G663, D665, D668, T670, H727, G733, and L746.

5. A bifunctional enzyme having γ-glutamylcysteine synthetase and glutathione synthetase activities.

6. A bifunctional enzyme of claim 5 where the enzyme is isolated from S. agalactiae.

7. A bifunctional enzyme of claim 5 where GSH does not inhibit either the γ-glutamylcysteine synthetase or the glutathione synthetase activity with a Kᵢ value of less than 100 mM.

8. A bifunctional enzyme of claim 5 which has a sequence identical to a sequence selected from the sequences set forth in the Sequence Listing as SEQ ID NO: 2, SEQ ID NO: 4, SEQ ID NO: 6, SEQ ID NO: 8, SEQ ID NO: 10, SEQ. ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, and SEQ ID NO: 28.

9. A bifunctional enzyme of claim 5 which has at least 27% sequence identity to any sequence selected from the group consisting of SEQ ID NO: 2, SEQ ID NO: 4, SEQ 1D NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, and SEQ ID NO: 28.

10. A bifunctional enzyme of claim 5 which additionally includes an N-terminal or C-terminal extension or both added to facilitate purification of the bifunctional enzyme.

11. A bifunctional enzyme of claim 10 in which the N-terminal or C-terminal extension (or both) are selected from the group comprising poly-histidine (His₃ to His₁₂), GSH S-transferase (GST), and maltose binding protein (MBP) with or without a linker susceptible to proteolytic cleavage.

12. A bifunctional enzyme of claim 11 in which the N-terminal extension is His₆ or His₈ and the remainder of the sequence corresponds to SEQ ID NO:2 or SEQ ID NO:12.

13. An expression vector containing a DNA molecule of claim 1.

14. A host cell containing an expression vector of claim 13.

15. A method for producing the bifunctional enzyme of claim 5 comprising the steps (a) culturing a host cell of claim 14, (b) breaking the cells to release intracellular proteins, and (c) purifying the γ-GCS-GS activity.

16. An inhibitor of the bifunctional enzyme of claim 5.

17. An inhibitor of the bifunctional enzyme of claim 5, the inhibitor having a molecular weight of less than or equal to about 750 Daltons, wherein the inhibitor is an analog of one or a plurality of substrates of the bifunctional enzyme.

18. An inhibitor of claim 16 that is an S-alkyl homocysteine sulfoximine inhibitor that inhibits the γ-GCS activity of the bifunctional enzyme.

19. An inhibitor of claim 18, wherein the S-alkyl homocysteine sulfoximine inhibitor has an S-alkyl group comprising I to 6 carbon atoms.

20. An inhibitor of claim 19, wherein the S-alkyl homocysteine sulfoximine inhibitor is L-buthionine-S-sulfoximine.

21. A method of treating a mammal suffering from an infection caused by an organism producing the bifunctional enzyme of claim 5 by administering to the host an inhibitor of the bifunctional enzyme.

22. The method of claim 21, wherein the inhibitor of the bifunctional enzyme is used in combination with a therapy for increasing oxidative stress in the infecting organism.

23. The method of claim 21, wherein the organism infecting the mammal is Streptococcus agalactiae, Streptococcus mutans, Streptococcus suis, Streptococcus thermophilus, Pasteurella multocida, Mannheimia succinicproducens, Haemophilus somnus, Enterococcus faecalis, Enterococcus faecium, Listeria monocytogenes, Listeria innocua, Clostridium perfringens, Lactobacillus plantarum or combinations of infecting organisms comprising at least one of the foregoing.

24. A method for producing glutathione from its constituent amino acids using the bifunctional enzyme of claim 5.

25. The method of claim 24, wherein at least one of the constituent amino acids is isotopically labeled.

26. The method of claim 25, wherein the isotopically labeled amino acid is ¹⁴C or ¹³C— or ³H—or ²H— or ¹⁵N— or ¹³N— or ¹⁷O— or ¹⁸O— or ³³S— or S-labeled L-glutamate, L-cysteine or glycine or mixtures thereof.

27. The method of claim 26, wherein the isotopically labeled amino acid(s) include at least one of L-[¹⁴C]glutamate, [¹⁴C]glycine, L-[³⁵S]cysteine, L-[¹³C]cysteine, L-[³H]glutamate or [¹⁵N]glycine.

28. The, method of claim 24, wherein a reaction mixture for producing glutathione includes an ATP-regenerating system.

29. A method for increasing glutathione levels in the cells of an organism the method comprising the transfection of the organism with a plasmid containing a DNA molecule of claims 1.

30. The method of claim 29 in which the DNA molecule encodes a protein having a sequence identical or substantially similar to the sequence of SEQ ID NO:2.

31. The method of claim 29 in which the plasmid causes the DNA molecule to be incorporated into the genome of the organism.

32. A method for identifying γ-GCS-GS inhibitors effective in reducing glutathione synthesis in intact bacteria comprising the steps (a) growing γ-GCS-GS-containing bacteria in wells of multi-well plates in which individual wells also contain any of a variety of compounds that are possible inhibitors, (b) optionally separating the bacteria from the media, (c) breaking the bacteria, (d) determining the amount of glutathione in the bacterial lysate, and (e) calculating for each well whether the amount of glutathione detected is reduced by the presence in that well of the compounds that are possible inhibitors, such reduction indentifying the compounds as inhibitors.

33. The method of claim 32 in which step (b) is achieved by centrifugation to sediment the bacteria and removal of the supernatant media, step (c) is achieved by addition of lysozyme or by sonication or by freeze-thawing, and step (d) is achieved by resuspending the bacterial lysate in a reaction mixture containing NADPH, glutathione disulfide reductase, and a disulfide that produces a chromaphore when reduced by glutathione, and monitoring the increase in chromaphore to identify those wells exhibiting reduced amounts of chromaphore due to inhibition of glutathione synthesis.
